SEQUENCE LISTING (15 sequences)

SEQ ID NO: 1 - DNA, 1074 bp, Homo sapiens
  source 1..1074: mol_type = genomic DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgttta

SEQ ID NO: 2 - protein, 289 aa, Homo sapiens
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP

SEQ ID NO: 3 - DNA, 1074 bp, synthetic construct
  source 1..1074: mol_type = other DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattagaccagttgccgtgagagttggtgggcgtcgccaggctgtggaatgtatcgaggacttgcttaacgagcctgggcaacctttggacttgagctgtagacgccccaggccataaggtgtaaacctgtgattgcgtgtgtggttaacgcctttgtttgctgaatgagttgatgtaagtttaataaagggtgagataatgttta

SEQ ID NO: 4 - protein, 289 aa, synthetic construct
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIRPVAVRVGGRRQAVECIEDLLNESGQPLDLSCRRPRP

SEQ ID NO: 5 - DNA, 253 bp, synthetic construct
  source 1..253: mol_type = other DNA
atgcgtgagtggctcgcccgcggacacgccagccccgccccgcgagcccggtttccccgccccctccccgcccacgccctgcgctcccttcctattggtcccatgccgcgctttcccgttcaatcgcagcgcttagcgccagaatttgaatcttcgttttcgtttgaattgggcgggcgcgccgggctggaagaaggaagtggagggctgacgctgcgggcgggcgggcgggcgggaggactcgactcggtgg

SEQ ID NO: 6 - DNA, 375 bp, synthetic construct
  source 1..375: mol_type = other DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactaca

SEQ ID NO: 7 - protein, 125 aa, synthetic construct
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 8 - DNA, 57 bp, synthetic construct
  source 1..57: mol_type = other DNA
gctactaacttcagcctgctgaagcaggctggagacgtggaggagaaccctggacct

SEQ ID NO: 9 - protein, 19 aa, synthetic construct
ATNFSLLKQAGDVEENPGP

SEQ ID NO: 10 - DNA, 662 bp, synthetic construct
  source 1..662: mol_type = other DNA
tcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcaaatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 11 - DNA, 54 bp, synthetic construct
  source 1..54: mol_type = other DNA
gagggcagaggaagtcttctaacatgcggtgacgtggaggagaatcccggccct

SEQ ID NO: 12 - protein, 18 aa, synthetic construct
EGRGSLLTCGDVEENPGP

SEQ ID NO: 13 - DNA, 987 bp, synthetic construct
  source 1..987: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 14 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 15 - DNA, 135 bp, synthetic construct
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

(19) United States
(12) Patent Application Publication
(10) Pub. No.: US 2025/0288628 A1
(43) Pub. Date: Sep. 18, 2025

(54) MUTANT E1A ANTAGONIZING UBIQUITINATION-MEDIATED DEGRADATION AND ITS USE IN THE PREPARATION OF POTENTIATED ONCOLYTIC ADENOVIRUSES AND IMMUNE POTENTIATORS

(71) Applicant: XUZHOU MEDICAL UNIVERSITY, Xuzhou (CN)

(72) Inventors: Gang WANG, Xuzhou (CN); Nan SUN, Xuzhou (CN); Junnian ZHENG, Xuzhou (CN); Lin FANG, Xuzhou (CN)

(73) Assignee: XUZHOU MEDICAL UNIVERSITY, Xuzhou (CN)

(21) Appl. No.: 19/207,415

(22) Filed: May 14, 2025

Related U.S. Application Data
(63) Continuation of application No. PCT/CN2023/112364, filed on Aug. 10, 2023.

(30) Foreign Application Priority Data
Dec. 29, 2022 (CN) 202211707030.7

(51) Int. Cl. A61K 35/761; A61P 35/00; C07K 14/005; C07K 14/52; C07K 14/54

(52) U.S. Cl. CPC A61K 35/761; A61P 35/00; C07K 14/005; C07K 14/522; C07K 14/5434; C12N 2710/10022; C12N 2710/10032; C12N 2710/10043; C12N 2710/10051

(57) ABSTRACT

A mutant E1A antagonizing ubiquitination-mediated degradation and its use in the preparation of potentiated oncolytic adenoviruses and immune potentiators are provided. The mutant E1A is obtained by mutating lysine residues at positions 253 and 285 of a wild-type E1A to any one of glycine, alanine, valine, leucine, isoleucine, proline, phenylalanine, tyrosine, tryptophan, serine, threonine, cysteine, methionine, asparagine, glutamine, aspartic acid, glutamic acid, arginine, or histidine. The mutation is capable of reducing the degradation of a key adenoviral replication protein E1A and maintaining its high level expression, thereby promoting replication of oncolytic adenovirus, improving tumor killing effect, and holding broad clinical application prospects.

### CROSS-REFERENCE TO RELATED APPLICATIONS

This application is a continuation of international application No. PCT/CN2023/112364, filed on Aug. 10, 2023, which claims priority to Chinese Patent Application No. 202211707030.7, filed on Dec. 29, 2022, the entire contents of each of which are incorporated herein by reference.

### SEQUENCE LISTING

The instant application contains a Sequence Listing which is submitted electronically in XML format and is hereby incorporated by reference in its entirety. The XML copy, created on Jun. 3, 2025, is named “2025 Jun. 3-Sequence Listing-6B803-H003US00,” and is 19,887 bytes in size.

### TECHNICAL FIELD

The present disclosure generally relates to the field of biomedical technology, and specifically to a mutant E1A antagonizing ubiquitination-mediated degradation and its use in the preparation of potentiated oncolytic adenoviruses and immune potentiators.

### BACKGROUND

Currently, cancer treatment mainly relies on chemotherapy, radiotherapy, and surgery. Although the cure rate for early-stage cancers has increased, the majority of advanced cancer cases are difficult to cure. In order to mitigate the situation, a variety of biotechnological strategies have been developed in the field of biomedical technology, aiming to improve the efficacy and selectivity of tumor treatment. Specifically, gene therapy and virotherapy utilize viruses to fight against cancer. In the gene therapy, viruses are modified to make them non-replicable and act as a mediator or a vector of therapeutic genetic material. However, the virotherapy utilizes viruses that are able to selectively replicate and proliferate in tumor cells. In the virotherapy, the death of tumor cells is not caused by therapeutic genes, but rather by cytopathic effects resulted from the replication of viruses inside the cells. Preferential replication in tumor cells is known as oncotropism, and tumor lysis is known as oncolysis. Oncolytic adenoviruses (OAVs) are a class of conditionally replicating adenoviruses. They can replicate relatively specifically in tumor cells and lyse tumor cells, release the progeny viruses to infect the surrounding tumor cells, and destroy the tumors through a cascade amplification effect, thus achieving a better therapeutic effect.

The OAVs is a novel therapy for treating tumors following surgery, radiotherapy, and chemotherapy. It can specifically recognize and infect tumor cells, ultimately leading to cell swelling and destruction of the tumor cells. Moreover, it cannot replicate within the cells of the normal organism and thus has no killing effect. Theoretically, the OAVs have a higher antiviral effect and a lower side effect, exhibiting good application prospects in preclinical and clinical studies. However, the OAVs still have certain limitations such as the need to further improvement of their lysogenic potency. The preliminary study demonstrated that the OAVs are also rejected by tumor cells when infecting tumor cells, and the ubiquitination-mediated degradation of E1A is an important mechanism for the rejection of the OAVs.

In view of the above, there is a need to provide a mutant E1A antagonizing ubiquitination-mediated degradation, which is capable of reducing the degradation of a key adenovirus replication protein E1A and maintaining its high-level expression, thus promoting the replication of the OAVs and improving the tumor killing effect.

SUMMARY

One or more embodiments of the present disclosure provides a mutant E1A antagonizing ubiquitination-mediated degradation, wherein the mutant E1A is obtained by mutating lysine residues at positions 253 and 285 of a wild-type E1A to any one of glycine, alanine, valine, leucine, isoleucine, proline, phenylalanine, tyrosine, tryptophan, serine, threonine, cysteine, methionine, asparagine, glutamine, aspartic acid, glutamic acid, arginine, or histidine.

One or more embodiments of the present disclosure provides an oncolytic adenovirus vector comprising the mutant E1A, wherein a backbone sequence of the oncolytic adenovirus vector is an adenovirus genome.

In some embodiments, a serotype of an adenovirus is Ad5, Ad3, Ad9, Ad11, Ad12, Ad34, or Ad37.

One or more embodiments of the present disclosure provides a potentiated oncolytic adenovirus vector comprising the mutant E1A, wherein the potentiated oncolytic adenovirus vector further includes: a sequence of at least one of an immunomodulatory factor, an immune checkpoint inhibitor, an immunomodulatory molecule, an antigenic molecule, an enzyme, or an small interfering RNA (siRNA).

In some embodiments, the immunomodulatory factor is selected from a group consisting of an interleukin (IL), an interferon (INF), a tumor necrosis factor, a colony stimulating factor, and a chemokine.

In some embodiments, the interleukin is selected from at least one of a group consisting of IL-1, IL-18, IL-33, IL-36, IL-37, IL-38, IL-2, IL-4, IL-13, IL-15, IL-21, IL-6, IL-11, IL-27, IL-31, OSM, LIF, CNTF, CT-1, CLCF1, IL-12, IL-23, IL-27, IL-35, IL-10, IL-19, IL-20, IL-22, IL-24, IL-26, IL-28, IL-29, IL-17, and IL-25.

In some embodiments, the interferon is selected from at least one of a group consisting of IFN-α, IFN-β, and IFN-γ.

In some embodiments, the chemokine is selected from at least one of a group consisting of CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8 (IL-8), CXCL9, CXCL10, CXCL11, CXCL12, CXCL13, CXCL14, CXCL15, CXCL16, CXCL17, XCL1, XCL2, and CX3CL1.

In some embodiments, the potentiated oncolytic adenovirus vector includes a sequence of the mutant E1A, a CXCL9 sequence, and an IL-12 sequence.

In some embodiments, a serotype of an adenovirus is Ad5, Ad3, Ad9, Ad11, Ad12, Ad34, or Ad37.

One or more embodiments of the present disclosure provides an oncolytic adenovirus, wherein the oncolytic adenovirus is obtained by packaging the oncolytic adenovirus vector.

One or more embodiments of the present disclosure provides an potentiated oncolytic adenovirus, wherein the potentiated oncolytic adenovirus is obtained by packaging the potentiated oncolytic adenovirus vector.

One or more embodiments of the present disclosure provides a pharmaceutical composition, comprising a therapeutically effective amount of the oncolytic adenovirus.

One or more embodiments of the present disclosure provides a pharmaceutical composition, comprising a therapeutically effective amount of the potentiated oncolytic adenovirus.

One or more embodiments of the present disclosure provides a biological agent, comprising a therapeutically effective amount of the oncolytic adenovirus and a pharmaceutically acceptable carrier or excipient.

One or more embodiments of the present disclosure provides a biological agent, comprising a therapeutically effective amount of the potentiated oncolytic adenovirus and a pharmaceutically acceptable carrier or excipient.

One or more embodiments of the present disclosure provides a method for inhibiting tumor progression or reducing tumor volume in a subject, or treating a tumor in the subject, comprising administering to the subject with a therapeutically effective amount of the oncolytic adenovirus, or a therapeutically effective amount of a pharmaceutical composition comprising the oncolytic adenovirus, or a therapeutically effective amount of a biological agent comprising the oncolytic adenovirus and a pharmaceutically acceptable carrier or excipient.

In some embodiments, the tumor is a lung cancer, a liver cancer, a myeloma, a thymoma, a sarcoma, a non-Hodgkin lymphoma, a Hodgkin lymphoma, a skin cancer, a uterine cancer, a breast cancer, a pancreatic cancer, a colorectal cancer, an anal cancer, a kidney cancer, a bladder cancer, a prostate cancer, an ovarian cancer, a brain cancer, hemangioendothelioma, a head and neck cancer, a thyroid cancer, a glioma, a testicular cancer, or a gastrointestinal cancer.

One or more embodiments of the present disclosure provides a method for inhibiting tumor progression or reducing tumor volume in a subject, or treating a tumor in the subject, comprising administering to the subject with a therapeutically effective amount of the potentiated oncolytic adenovirus, a therapeutically effective amount of a pharmaceutical composition comprising the potentiated oncolytic adenovirus, or a therapeutically effective amount of a biological agent comprising the potentiated oncolytic adenovirus and a pharmaceutically acceptable carrier or excipient.

In some embodiments, the tumor is a lung cancer, a liver cancer, a myeloma, a thymoma, a sarcoma, a non-Hodgkin lymphoma, a Hodgkin lymphoma, a skin cancer, a uterine cancer, a breast cancer, a pancreatic cancer, a colorectal cancer, an anal cancer, a kidney cancer, a bladder cancer, a prostate cancer, an ovarian cancer, a brain cancer, hemangioendothelioma, a head and neck cancer, a thyroid cancer, a glioma, a testicular cancer, or a gastrointestinal cancer.

### BRIEF DESCRIPTION OF THE DRAWINGS

The present disclosure is further described in terms of exemplary embodiments, these exemplary embodiments are described in detail with reference to the drawings. These embodiments are non-limiting exemplary embodiments, in which like reference numerals represent similar structures throughout the several views of the drawings, wherein:

FIGS. 1A-1B are schematic diagrams of results illustrating that amino acid mutations at positions 253 and 285 in a key adenoviral replication gene E1A reduce its ubiquitination-mediated degradation according to some embodiments of the present disclosure, FIG. 1A shows a nucleotide sequence of E1A, with the bolded parts as the mutation sites; and FIG. 1B shows an assay of E1A ubiquitination-mediated degradation (E1A point mutation antagonizing the ubiquitination-mediated degradation induced by E3 ubiquitin ligase TRIM35);

FIGS. 2A-2F are schematic diagrams of cellular morphological changes and lesions after 24 h of infection with OAV and OAV/E1A(K253/285R) in different cell lines according to some embodiments of the present disclosure, FIG. 2A shows the 7860 cell line; FIG. 2B shows the OSRC cell line; FIG. 2C shows the PC-3 cell line; FIG. 2D shows the U251 cell line; and FIG. 2E shows the U87 cell line; and FIG. 2F shows the RM-1 cell line;

FIGS. 3A-3D are tumor killing curves of OAV and OAV/E1A(K253/285R) in different cell lines detected by real-time cell analysis (RTCA) according to some embodiments of the present disclosure, FIG. 3A shows the 7860 cell line; FIG. 3B shows the PC-3 cell line; FIG. 3C shows the OSRC cell line; and FIG. 3D shows the U251 cell line;

FIGS. 4A-4E are schematic diagrams of the results illustrating that amino acid mutations at positions 253 and 285 of the key adenovirus replication gene E1A enhance in vivo tumor killing effect of the oncolytic adenovirus according to some embodiments of the present disclosure, FIG. 4A shows the tumor growth curves of the mice in three groups (PBS, OAV, and OAV/E1A(K253/285R)); FIG. 4B shows the tumor inhibition rate; FIG. 4C shows the weight of the heaviest transplanted tumors in mice; FIG. 4D shows the photographs of the transplanted tumors of mice in three groups; FIG. 4E shows the schematic diagram of hematoxylin and eosin (HE) staining of important organs including heart, liver, spleen, lung, and kidney;

FIG. 5A-5B are schematic diagrams of the contents of CXCL9 and IL-12 in the supernatants after OAV/E1A(K253/285R)-CXCL9+IL12 infects different target cells detected by enzyme-linked immunosorbent assay (ELISA) according to some embodiments of the present disclosure, FIG. 5A shows CXCL9; and FIG. 5B shows IL-12;

FIG. 6 is a schematic diagram of directional chemotaxis of the supernatants from liver cancer target cells infected with OAV/E1A(K253/285R)-CXCL9+IL12 to activated T cells detected by Transwell according to some embodiments of the present disclosure, where blank is normal cultured activated T cells, CXCL9 is normal cultured activated T cells with addition of CXCL9 (10 ng/ml), Tumor cells-CM is a culture supernatant of Huh7 cells, and tumor cells infected with OAV-CM is a supernatant of Huh7 cells after being infected with Ad5-Ki67-ZD55-BC mutant-CXCL9+IL12;

FIG. 7 is a schematic diagram of the promoting effect of supernatants from liver cancer target cells infected with OAV/E1A(K253/285R)-CXCL9+IL12 to proliferation of activated T cell detected by flow cytometry according to some embodiments of the present disclosure, where control is cytokine-starved T cells, IL12 (20 ng/ml) is cytokine-starved T cells with addition of IL12 (20 ng/mL), Huh7-CM is a culture supernatant of Huh7 cells, Huh7-OAV-CXCL9-IL12-CM is a supernatant of Huh7 cells after being infected with Ad5-Ki67-ZD55-BC mutant-CXCL9+IL12, A549-CM is a culture supernatant of A549 cells, A549-OAV-CXCL9-IL12-CM is a supernatant of A549 cells after being infected with Ad5-Ki67-ZD55-BC mutant-CXCL9+IL 12;

FIG. 8 is a schematic diagram of promotion of IFN-γ expression in T cells by supernatants from liver cancer target cells infected with OAV/E1A(K253/285R)-CXCL9+IL12 detected by flow cytometry according to some embodiments of the present disclosure, where control is cytokine -starved T cells, IL12 (20 ng/ml ) is cytokine -starved T cells with in addition of IL12 (20 ng/ml ), Huh7 -CM is a culture supernatant of Huh7 cells, Huh7 -OAV -CXCL9 -IL12 -CM is a supernatant of Huh7 cells after being infected with Ad5 -Ki67 -ZD55 -BC mutant -CXCL9+IL12, A549 -CM is a culture supernatant of A549 cells, A549 -OAV -CXCL9 -IL12 -CM is a supernatant of A549 cells after being infected with Ad5 -Ki67 -ZD55 -BC mutant -CXCL9+IL12; and

FIGS. 9A-9C are schematic diagrams of an oncolytic adenovirus (OAV) of a wild-type E1A, an oncolytic adenovirus (OAV/E1A(K253/285R)) of K253/285R mutant E1A, and an oncolytic adenovirus (OAV/E1A(K253/285R)-CXCL9+IL12) co-expressing CXCL9 and IL12 according to some embodiments of the present disclosure, FIG. 9A shows OAV; FIG. 9B shows OAV/E1A(K253/285R); and FIG. 9C shows OAV/E1A(K253/285R)-CXCL9+IL12.

### DETAILED DESCRIPTION

All technical and scientific terms used in the present disclosure have the same meanings as those commonly understood by a person of ordinary skill in the art to which the present disclosure belongs, unless otherwise defined. In order to facilitate understanding of the present disclosure, the following terms involved in the present disclosure are explained herein.

The present disclosure identifies a mutant E1A antagonizing ubiquitination-mediated degradation, i.e., mutating lysine residues at positions 253 and 285 of a wild-type E1A to arginine (K253/285R). This mutation can reduce the degradation of the key adenoviral replication protein E1A, thereby promoting the replication of oncolytic adenovirus and improving tumor killing effect. In addition, the mutant E1A is obtained by mutating the lysine residue at position 253 of a wild-type E1A to any one of glycine, alanine, valine, leucine, isoleucine, proline, phenylalanine, tyrosine, tryptophan, serine, threonine, cysteine, methionine, asparagine, glutamine, aspartic acid, glutamic acid, arginine, or histidine, and mutating the lysine residue at position 285 of a wild-type E1A to any one of glycine, alanine, valine, leucine, isoleucine, proline, phenylalanine, tyrosine, tryptophan, serine, threonine, cysteine, methionine, asparagine, glutamine, aspartic acid, glutamic acid, arginine, or histidine. The obtained mutant E1A can also reduce the degradation of key adenoviral replication protein E1A, thereby promoting the replication of oncolytic adenovirus and enhancing tumor killing effect. Thus, the mutant E1A obtained by mutating lysine residues at positions 253 and 285 of the wild-type E1A to any amino acid other than lysine is within the scope of protection of the present disclosure.

As used herein, the terms “including” or “comprising” refer to including the stated elements or components and does not exclude other elements or components.

As used herein, the terms “the oncolytic adenovirus” and “the potentiated oncolytic adenovirus” refer to a class of adenoviruses that are genetically engineered. These adenoviruses can selectively replicate in tumor cells and lyse and kill tumor cells, while not affecting function of normal cells. The oncolytic adenovirus and the potentiated oncolytic adenovirus provided in the present disclosure have a broad-spectrum applicability to tumors. In specific embodiments of the present disclosure, in vivo and in vitro experimental tests are conducted on numerous tumor cells, and the results confirm that the oncolytic adenovirus and the potentiated oncolytic adenovirus have broad-spectrum tumor killing effect. The main mechanism of the tumor killing is to prevent E1A from ubiquitination-mediated degradation by introducing two mutation sites in the adenovirus E1A, thereby reducing the degradation of the key adenoviral replication protein E1A and maintaining its high-level expression, which in turn promotes the replication of the oncolytic adenovirus and improves the tumor killing effect. In addition, the exogenous gene expressed by the potentiated oncolytic adenovirus after infecting tumor cells can significantly induce directional chemotaxis of T cells, promote the proliferation of T cells, and promote the expression of IFN-γ in T cells, thereby improving the tumor killing effect.

In selecting or designing therapeutic oncolytic adenovirus with desired therapeutic activity, a variety of biological properties are considered, including: selectively targeting cancer cells for infection through the natural orientation of cell-surface proteins or by engineering adenovirus to directly target cancer cells; selective replication in cancer cells; attenuation of viral pathogenicity; enhancement of lysis activity; modification of the antiviral immune response leading to rapid clearance of adenovirus; and modification of systemic anti-tumor immunity through incorporating cytokines, immune agonists, or immune checkpoint blockers by genetic modification of the adenovirus. The oncolytic adenovirus vector with replication ability has several properties including infectivity in various cells and tumors, infectivity in non-dividing cells, lack of genomic integration, high titers, ability to carry transgenes, in vitro and in vivo stability, and transgene expression, making it an ideal choose for therapeutic application. Adenovirus expression vector includes constructs including adenovirus sequences that is sufficient to support packaging of the constructs and eventual expression of recombinant gene constructs that have been cloned therein.

In some embodiments, a treatment regimen using the oncolytic adenovirus or the potentiated oncolytic adenovirus of the present disclosure include a single administration or a plurality of administrations. A plurality of administrations may be performed according to a periodic schedule and/or in response to one or more indicators of efficacy of one or more previous administrations, or side effects of one or more previous administrations, as well as other indicators that are obvious to those of ordinary skill in the art who benefit from this present disclosure. When administered to a subject, an effective amount of a composition described herein is given for the treatment (e.g., palliation, amelioration, mitigation, reduction, stabilization, prevention of spread, slowing or delaying the progression of cancer, or curing). For example, the dosage is sufficient to achieve the effect of reducing or destroying cancer cells or tumor cells or inhibiting the growth and/or proliferation of such cells. In order to achieve the desired clinical efficacy, the compositions provided herein are administered one or more times according to the treatment regimen.

As used herein, the term “promoter” refers to a DNA sequence capable of driving the expression of a target gene. The appropriate promoters of the present disclosure include promoters from mammalian viruses and promoters from mammalian cell genomes, including, but not limited to, cytomegalovirus (CMV) promoter, U6 promoter, H1 promoter, murine leukemia virus (MLV) long terminal repeat (LTR) promoter, adenovirus early promoter, adenovirus late promoter, vaccinia virus 7.5K promoter, SV40 promoter, HSV tk promoter, RSV promoter, EF1α promoter, methallothionin promoter, β-actin promoter, human IL-2 gene promoter, human IFN gene promoter, human IL-4 gene promoter, human lymphotoxin gene promoter, human GM-CSF gene promoter, human phosphoglycerate kinase (PGK) promoter, mouse phosphoglycerate kinase (PGK) promoter and survivin promoter.

As used herein, the term “a therapeutically effective amount” refers to the amount of the oncolytic adenovirus, the potentiated oncolytic adenovirus, the pharmaceutical composition, and/or the biological agent that can effectively inhibit, prevent, stop, delay, or treat specific symptoms of diseases, conditions, or side effects. The therapeutically effective amount may vary as a function of different variables such as the mode of administration, the disease state, the age and weight of the subject, the ability of the subject to respond to the treatment, the type of concurrent treatment, the frequency of treatment, and/or the need for treatment or prevention. When used in the prevention of disease, the oncolytic adenovirus, the potentiated oncolytic adenovirus, the pharmaceutical composition, and/or the biological agent are administered in a subject at risk of disease at a dosage sufficient to prevent or delay the occurrence and/or establishment and/or recurrence of a pathological condition (e.g., the tumor). When used in the treatment of a disease, the therapeutically effective amount is a dosage that results in a visible improvement in clinical status compared to the baseline or expected state without treatment, such as reduction in the number of tumors, reduction in the size of tumors, reduction in the number or extent of metastases, prolongation of remission period, stabilization of the disease state (i.e., no worsening), delaying or slowing down of the progression or severity of the disease, improvement or alleviation of the disease state, prolongation of survival time, better response to conventional treatment, improvement in the quality of life, and reduction in mortality.

The therapeutically effective amount is also a necessary dosage to bring about the development of an effective non-specific (innate) and/or specific anti-tumor response. Typically, the development of an immune response within a specific T cell response may be assessed in vitro, within a suitable animal model, or using biological samples collected from a subject. For example, technical tools commonly used in the laboratory (e.g., flow cytometry, histology) are used to monitor tumors. A plurality of existing antibodies may also be used to identify different immune cell populations present in the subject that are involved in anti-tumor responses, such as cytotoxic T cells, activated cytotoxic T cells, natural killer cells, and activated natural killer cells. Improvement in clinical status can be readily assessed by relevant clinical measurements used by any doctor or other healthcare technician.

As used herein, the term “a pharmaceutically acceptable carrier or excipient” refers to a substance that is suitable for use in humans and/or mammals without undue adverse side effects (e.g., toxicity, irritation, and anaphylaxis), i.e., a substance with a reasonable benefit/risk ratio. The pharmaceutically acceptable carrier or excipient used in the present disclosure are conventional, and Remington's Pharmaceutical Sciences, Mack Publishing Co. of Easton, PA, 15th edition (1975) written by E.W. Martin describes compositions and formulations suitable for the compositions and formulations for drug delivery of one or more therapeutic compounds, molecules, or reagents (e.g., the oncolytic adenovirus or the potentiated oncolytic adenovirus as disclosed herein). Usually, the nature of the carrier depends on the employed particular administration mode. For example, parenteral formulations typically include injectable fluids as carriers, including pharmaceutically and physiologically acceptable fluids, such as water, saline, a balanced salt solution, an aqueous dextrose solution, glycerol, etc. For solid compositions (e.g., in the form of powder, pill, tablet, or capsule), conventional non-toxic solid carriers include, for example, pharmaceutical grade mannitol, lactose, starch, or magnesium stearate. In addition to the bio-neutral carrier, the pharmaceutical composition to be administered contains small amounts of non-toxic auxiliary substances, such as wetting agents or emulsifiers, preservatives, and pH buffering agent, such as sodium acetate or dehydrated sorbitol monolaurate.

As used herein, the term “the pharmaceutical composition” or “the biological agent” have any one of the following formulations: tablets, pills, powders, granules, capsules, suspensions, solutions, emulsions, syrups, sterilized aqueous solutions, non-aqueous solutions, suspending formulations, emulsions, lyophilized formulations, and suppositories. In addition, it may be administered one or more times. At this time, the pharmaceutical composition and/or the biological agent is administered in the form of a liquid formulation, a powder, an aerosol, a capsule, a vaginal tablet, a capsule, or a suppository.

In some embodiments, the route of administration includes, but is not limited to: intraperitoneal, intravenous, intramuscular, subcutaneous, intradermal, oral, topical, intranasal, intrapulmonary, intrarectally, etc. When administered orally, it may be formulated into a coating that protects the active ingredients in the pharmaceutical composition from degradation in the stomach. Additionally, the active ingredient is administered by any device capable of transferring to the target tissue. In some embodiments, the pharmaceutical composition provided herein is made in a plurality of dosage forms as desired and the dosage beneficial to the subject is determined by a clinician based on the type of the subject, age of the subject, weight of the subject, and approximate disease status of the subject, the mode of administration, and other factors. The mode of administration is, for example, injection or any other suitable mode of administration known to those skilled in the art.

In the present disclosure, the oncolytic adenovirus, the potentiated oncolytic adenovirus, the pharmaceutical composition, or the biological agent are used concurrently or sequentially in combination with other anticancer therapies. The other anticancer therapies include, but are not limited to, chemotherapeutic agents, targeted anticancer agents, radiotherapy, radioisotope therapy, or any combination thereof. In some embodiments, the chemotherapeutic agents refer to specific anti-tumor chemical agents or drugs that selectively destroy malignant cells and tissues. For example, alkylating agents, antimetabolites, anthracyclines, plant alkaloids, topoisomerase inhibitors, and other anti-tumor agents. In some embodiments, the chemotherapeutic agents also include doxorubicin, 5-fluorouracil (5-FU), paclitaxel, capecitabine, irinotecan, and platinum compounds such as cisplatin and oxaliplatin. In some embodiments, the dosage is selected by a professional person based on the nature of the cancer. In some embodiments, the targeted anticancer agents refer to drugs that specifically attacks a target or several targets among malignant tumor cells to achieve the effect of inhibiting the proliferation of tumor cells. For example, gefitinib, erlotinib, icotinib, afatinib, osimertinib, almonertinib, anlotinib, crizotinib, ceritinib, alectinib, bevacizumab, recombinant human endostatin, cetuximab, bevacizumab, regorafenib, fruquintinib, imatinib, sunitinib, trastuzumab, apatinib, pazopanib, axitinib, sorafenib, everolimus, trastuzumab, inetetamab, pertuzumab, pyrotinib, olaparib, niraparib, enzalutamide, lenvatinib; nimotuzumab, vemurafenib, trametinib, dabrafenib, dasatinib, flumatinib, nilotinib, ibrutinib, zanubrutinib, ruxolitinib, azacitidine, rituximab, chidamide. In some embodiments, the radiotherapy refers to the use of a strong energy beam to kill cancer cells. Radiotherapy most often uses X-rays, but other types of radiation beam may also be used, including proton radiation. Based on the type and location of cancer, radiation oncologists use two types of radiotherapy either alone or in combination, including external radiotherapy (teletherapy) and internal radiotherapy (brachytherapy). In some embodiments, the radioisotope therapy refers to a tumor treatment method for destroying abnormal cells using radioisotopes that emit high-energy alpha and beta radiation. Radioisotopes commonly used in the tumor treatment include but are not limited to radioactive iodine, radioactive cesium, radioactive iridium, and radioactive cobalt.

As used herein, the term “subject” includes both human beings and non-human animals. The non-human animals include all vertebrates (e.g., mammals and non-mammals) such as non-human primates (e.g., cynomolgus monkeys), sheep, dogs, cattle, chickens, amphibians, and reptiles. In some embodiments of the present disclosure, the “subject” is a human.

In order to overcome the current technical problems in the field, the present disclosure provides a mutant E1A antagonizing ubiquitination-mediated degradation and its use in the preparation of potentiated oncolytic adenoviruses and immune potentiators.

One or more embodiments of the present disclosure provides the mutant E1A antagonizing ubiquitination-mediated degradation.

In some embodiments, the mutant E1A is obtained by mutating lysine residues at positions 253 and 285 of a wild-type E1A.

In some embodiments, the mutant E1A is obtained by mutating lysine residues at positions 253 and 285 of the wild-type E1A to any one of glycine, alanine, valine, leucine, isoleucine, proline, phenylalanine, tyrosine, tryptophan, serine, threonine, cysteine, methionine, asparagine, glutamine, aspartic acid, glutamic acid, arginine, or histidine.

In some embodiments, the mutant E1A is obtained by mutating lysine residues at positions 253 and 285 of the wild-type E1A to arginine.

In some embodiments, the wild-type E1A has a Gene Identifier (ID) of 2652980 in National Center for Biotechnology Information (NCBI). An amino acid sequence of the wild-type E1A is shown in SEQ ID NO:2, and a nucleotide sequence encoding the wild-type E1A is shown in SEQ ID NO:1. In some embodiments, an amino acid sequence of the mutant E1A is shown in SEQ ID NO:4, and a nucleotide sequence encoding the mutant E1A is shown in SEQ ID NO:3.

One or more embodiments of the present disclosure provides an oncolytic adenovirus vector.

In some embodiments, a backbone sequence of the oncolytic adenovirus vector is an adenovirus genome, and the E1A sequence is modified, the modification including: mutations of lysine residues at positions 253 and 285 of the E1A.

In some embodiments, lysine residues at positions 253 and 285 of the E1A are mutated to any one of glycine, alanine, valine, leucine, isoleucine, proline, phenylalanine, tyrosine, tryptophan, serine, threonine, cysteine, methionine, asparagine, glutamine, aspartic acid, glutamic acid, arginine, or histidine

In some embodiments, lysine residues at positions 253 and 285 of the E1A are mutated to arginine.

In some embodiments, a serotype of the adenovirus is Ad5, Ad3, Ad9, Ad11, Ad12, Ad34, or Ad37.

In some embodiments, the serotype of the adenovirus is Ad5.

One or more embodiments of the present disclosure provide the potentiated oncolytic adenovirus vector.

In some embodiments, the potentiated oncolytic adenovirus vector includes:
(i) a sequence of the mutant E1A as described above; and
(ii) a sequence of at least one of an immunomodulatory factor, an immune checkpoint inhibitor, an immunomodulatory molecule, an antigenic molecule, an enzyme, or an small interfering RNA (siRNA).

In some embodiments, the immunomodulatory factor is selected from a group consisting of an interleukin (IL), an interferon (INF), a tumor necrosis factor, a colony stimulating factor, and a chemokine.

In some embodiments, the interleukin is selected from at least one of a group consisting of IL-1, IL-18, IL-33, IL-36, IL-37, IL-38, IL-2, IL-4, IL-13, IL-15, IL-21, IL-6, IL-11, IL-27, IL-31, OSM, LIF, CNTF, CT-1, CLCF1, IL-12, IL-23, IL-27, IL-35, IL-10, IL-19,IL-20, IL-22, IL-24, IL-26, IL-28, IL-29, IL-17, and IL-25. In some embodiments, the interferon is selected from at least one of a group consisting of IFN-α, IFN-β, and IFN-γ. In some embodiments, the tumor necrosis factor is selected from at least one of a group consisting of IFN-α and IFN-β. In some embodiments, the colony stimulating factor is selected from at least one of a group consisting of G-CSF, M-CSF, GM-CSF, Multi-CSF (IL-3), SCF, and EPO. In some embodiments, the chemokine is selected from at least one of a group consisting of CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8 (IL-8), CXCL9, CXCL10, CXCL11, CXCL12, CXCL13, CXCL14, CXCL15, CXCL16, CXCL17, XCL1, XCL2, and CX3CL1.

In some embodiments, the immune checkpoint inhibitor includes polyclonal antibodies, monoclonal antibodies, ScFv antibodies, and bispecific antibody molecules against immune checkpoint molecules. In some embodiments, the immune checkpoint molecules are selected from at least one of a group consisting of CTLA-4, PD-1, PD-L1, LAG3, TIGIT, TIM3, B7H3, CD39, CD73, adenosine A2A receptor, Siglec-10, SIRPα, CD24, CD155, and CD47. In some embodiments, the immunomodulatory molecule is selected from at least one of a group consisting of 4-1BB (CD137), OX40, GITR, CD40, B7-1, B7-2, MHCI, and MHCII. In some embodiments, the antigenic molecule is selected from at least one of a group consisting of MUC1, WT1, E7, MAGE-A1, MAGE-A3, Claudin6, HPV E6, HPV E7, NY-ESO-1, EpCAM, ROR1, HER2, CD19, CD20, CD33, CD123, PSMA, Mesothelin, FBP, EGFRvlll, GD2, and survivin. In some embodiments, the enzyme includes thymidine kinase, cytosine deaminase, and/or hyaluronidase. In some embodiments, the siRNA is siRNA of a pathogenic gene or an immunosuppressive gene.

In some embodiments, the oncolytic adenovirus vector includes a sequence of the mutant E1A, a CXCL9 sequence, and an IL-12 sequence.

In some embodiments, the serotype of the adenovirus is Ad5, Ad3, Ad9, Ad11, Ad12, Ad34, or Ad37. In some embodiments, the serotype of the adenovirus is Ad5.

In some embodiments, the adenovirus is not limited to adenovirus with the serotype of Ad5. Currently, more than 100 serotypes discovered in terms of adenovirus serotype selection are within the scope of protection of the present disclosure. Specifically, there are 52 human adenoviruses, which are divided into six subgroups A, B, C, D, E, and F, and the human adenoviruses have different tropism, tumorigenicity, and disease histories towards host cells. The commonly used human adenovirus serotypes 2 and 5 in gene therapy belong to the subgroup C in serology, with 95% homology in their DNA sequence. The adenovirus vectors of the subgroup C have been successfully applied in clinical practice, and their safety has been widely tested in humans. As a result, these adenovirus vectors have high safety. In some embodiments, the serotype of the adenovirus is Ad5.

One or more embodiments of the present disclosure provide an oncolytic adenovirus.

In some embodiments, the oncolytic adenovirus is obtained by packaging the oncolytic adenovirus vector.

In some embodiments, the oncolytic adenovirus operably includes the following elements linked in sequence: a Ki67 promoter, the mutant E1A, Ad5 Hexon, and Ad5 Fiber. The amino acid sequence of the mutant E1A is shown in SEQ ID NO:4, the nucleotide sequence encoding the mutant E1A is shown in SEQ ID NO:3; and the nucleotide sequence of the Ki67 promoter is shown in SEQ ID NO:5.

One or more embodiments of the present disclosure provide a potentiated oncolytic adenovirus.

In some embodiments, the potentiated oncolytic adenovirus is obtained by packaging the potentiated oncolytic adenovirus vector.

In some embodiments, the oncolytic adenovirus operably includes the following elements linked in sequence: a Ki67 promoter, the mutant E1A, Ad5 Hexon, a CMV promoter, CXCL9, P2A, IL-12A, T2A, IL-12B, PolvA (SV40 polyA), and Ad5 Fiber. The amino acid sequence of the mutant E1A is shown in SEQ ID NO:4, and the nucleotide sequence encoding the mutant E1A is shown in SEQ ID NO:3. The nucleotide sequence of the Ki67 promoter is shown in SEQ ID NO:5. The amino acid sequence of CXCL9 is shown in SEQ ID NO:7, and the nucleotide sequence encoding CXCL9 is shown in SEQ ID NO:6. The amino acid sequence of P2A is shown in SEQ ID NO:9, and the nucleotide sequence encoding P2A is shown in SEQ ID NO. 8. The nucleotide sequence of IL-12A is shown in SEQ ID NO: 10. The amino acid sequence of T2A is shown in SEQ ID NO: 12, and the nucleotide sequence encoding T2A is shown in SEQ

ID NO: 11. The amino acid sequence of IL-12B is shown in SEQ ID NO: 14, and the nucleotide sequence encoding IL-12B is shown in SEQ ID NO:13. The nucleotide sequence of SV40 polyA is shown in SEQ ID NO: 15.

One or more embodiments of the present disclosure provide a pharmaceutical composition.

In some embodiments, the pharmaceutical composition includes a therapeutically effective amount of the oncolytic adenovirus and/or the potentiated oncolytic adenovirus.

One or more embodiments of the present disclosure provide a biological agent.

In some embodiments, the biological agent includes a therapeutically effective amount of the oncolytic adenovirus and a pharmaceutically acceptable carrier or excipient. In some embodiments, the biological agent includes a therapeutically effective amount of the potentiated oncolytic adenovirus and a pharmaceutically acceptable carrier or excipient.

In some embodiments, a suitable administration dosage of the pharmaceutical composition or the biological agent may be prescribed in a variety of ways according to factors such as the method of formulation, the mode of administration, the age, weight, gender, medical condition, diet, time of administration, route of administration, rate of excretion, and responsiveness of the subject. The physician can often easily determine the prescription and the corresponding administration dosage that is effective for the desired treatment. In some embodiments, the pharmaceutical composition or the biological agent is administered by any suitable method and delivery system known in the art, such as push infusion, injection such as intravenous or subcutaneous injection. In some embodiments, the pharmaceutical composition or the biological agent is administered by parenteral administration, intrapulmonary and intranasal administration, and intra-lesional (if desired for local treatment) administration. Parenteral infusion includes intramuscular administration, intravenous administration, intra-arterial administration, intraperitoneal administration, or subcutaneous administration.

One or more embodiments of the present disclosure provide a kit for the preparation of the oncolytic adenovirus.

In some embodiments, the kit includes:
(i) the oncolytic adenovirus vector and/or the potentiated oncolytic adenovirus vectors described above; and
(ii) virus-producing cells.

In some embodiments, the kit includes at least one of the oncolytic adenoviruses as described above in a container (e.g., in a sterile glass or plastic vial).

In some embodiments, the kit includes a device for administering the active ingredient. In some embodiments, the kit further includes an instruction manual, which includes information related to the constituent ingredients of the kit, or portions thereof, and usage instructions.

One or more embodiments of the present disclosure provide a method for preparing the oncolytic adenovirus vector and/or the potentiated oncolytic adenovirus as described above.

In some embodiments, the method includes:
(i) preparing the oncolytic adenovirus vector and/or the potentiated oncolytic adenovirus vector as described above;
(ii) transfecting the virus-producing cells with the oncolytic adenovirus vector and/or the potentiated oncolytic adenovirus vector prepared in step (i) and packaging to obtain the oncolytic adenovirus and/or the potentiated oncolytic adenovirus.

In some embodiments, the oncolytic adenovirus or the potentiated oncolytic adenovirus of the present disclosure is obtained by conventional preparation methods known to those skilled in the art, and the preparation methods include, but are not limited to, the following steps: preparation of the oncolytic adenovirus vector, cell transfection, amplification and virus harvesting, mass amplification and virus harvesting, treatment before purification, purification and concentration, and viral titer assay.

One or more embodiments of the present disclosure provide a method for inhibiting the viability of a tumor cell.

In some embodiments, the method includes: contacting the tumor cell with the oncolytic adenovirus as described above, the potentiated oncolytic adenovirus as described above, the pharmaceutical composition as described above, the biological agent as described above, and/or the test kit as described above.

In some embodiments, the method is an in vitro method.

In some embodiments, the method is an in vivo method, and contacting the tumor cell includes administering to the subject with the tumor with a therapeutically effective amount of the oncolytic adenovirus as described above, a therapeutically effective amount of the potentiated oncolytic adenovirus as described above, a therapeutically effective amount of the pharmaceutical compositions as described above, and/or a therapeutically effective amount of the biological agents as described above.

One or more embodiments of the present disclosure provide a method for inhibiting tumor progression or reducing tumor volume in the subject, or treating a tumor in the subject.

In some embodiments, the method includes administering to the subject with a therapeutically effective amount of the oncolytic adenovirus, or a therapeutically effective amount of a pharmaceutical composition comprising the oncolytic adenovirus, or a therapeutically effective amount of a biological agent comprising the oncolytic adenovirus and a pharmaceutically acceptable carrier or excipient, thereby inhibiting tumor progression or reducing tumor volume in the subject, or treating the tumor in the subject.

In some embodiments, the tumor includes cancers of the head, the neck, eyes, the mouth, the esophagus, the trachea, the larynx, the pharynx, the thorax, bones, lungs, the colon, the rectum, the stomach, the prostate, the bladder, the uterus, the cervix, the breast, the ovary, the testis, skins, the thyroid, blood, the lymph node, the kidney, the liver, the pancreas, the brain, or central nervous system.

In some embodiments, the tumor is a lung cancer, a liver cancer, a myeloma, a thymoma, a sarcoma, a non-Hodgkin lymphoma, a Hodgkin lymphoma, a skin cancer, a uterine cancer, a breast cancer, a pancreatic cancer, a colorectal cancer, an anal cancer, a kidney cancer, a bladder cancer, a prostate cancer, an ovarian cancer, a brain cancer, hemangioendothelioma, a head and neck cancer, a thyroid cancer, a glioma, a testicular cancer, or a gastrointestinal cancer.

In some embodiments, the method further includes administering to the subject with a therapeutically effective amount of the potentiated oncolytic adenovirus, a therapeutically effective amount of a pharmaceutical composition comprising the potentiated oncolytic adenovirus, or a therapeutically effective amount of a biological agent comprising the potentiated oncolytic adenovirus and a pharmaceutically acceptable carrier or excipient, thereby inhibiting tumor progression or reducing tumor volume in the subject, or treating the tumor in the subject.

In some embodiments, the tumor includes cancers of the head, the neck, eyes, the mouth, the esophagus, the trachea, the larynx, the pharynx, the thorax, bones, lungs, the colon, the rectum, the stomach, the prostate, the bladder, the uterus, the cervix, the breast, the ovary, the testis, skins, the thyroid, blood, the lymph node, the kidney, the liver, the pancreas, the brain, or central nervous system.

In some embodiments, the tumor is the lung cancer, the liver cancer, the myeloma, the thymoma, the sarcoma, the non-Hodgkin lymphoma, the Hodgkin lymphoma, the skin cancer, the uterine cancer, the breast cancer, the pancreatic cancer, the colorectal cancer, the anal cancer, the kidney cancer, the bladder cancer, the prostate cancer, the ovarian cancer, the brain cancer, hemangioendothelioma, the head and neck cancer, the thyroid cancer, the glioma, the testicular cancer, or the gastrointestinal cancer.

In some embodiments, the mode of administration includes any conventional route of administration, including, but not limited to, parenteral, topical, or mucosal routes. The parenteral route refers to administration by injection or infusion, including systemic and topical routes. The common types of parenteral injections are intravenous (into veins), intra-arterial (into arteries), intradermal (into the dermis), subcutaneous (below the epidermis), intramuscular (into muscles), and intratumoral (into the tumor or a site extremely close to the tumor). Typical infusion is performed via the intravenous route. Mucosal administration includes, but is not limited to, oral/esophageal, intranasal, endotracheal, intrapulmonary, intravaginal, or intrarectal routes. Topical administration may also be accomplished by transdermal means (e.g., patches, etc., etc.). Conventional syringes and needles (e.g., Quadrafuse injection needles) or any compound or device available in the prior art that facilitates or enhances delivery of the active agent within the subject are used to administer. In some embodiments, the routes for administering the oncolytic adenovirus include both intravenous and intratumoral routes.

One or more embodiments of the present disclosure provide the use of the mutant E1A in the preparation of the oncolytic adenovirus vector, the potentiated oncolytic adenovirus vector, the oncolytic adenovirus, the potentiated oncolytic adenovirus, the immune potentiator, the anti-tumor drugs, and the anti-tumor biological agent.

One or more embodiments of the present disclosure provide the use of the oncolytic adenovirus vector in the preparation of the oncolytic adenovirus, the anti-tumor drug, the anti-tumor biological agent, and the immune potentiator.

One or more embodiments of the present disclosure provide the use of the potentiated oncolytic adenovirus vector in the preparation of the potentiated oncolytic adenovirus, the anti-tumor drug, the anti-tumor biological agent, and the immune potentiator.

One or more embodiments of the present disclosure provide the use of the kit in the preparation of the oncolytic adenovirus, the potentiated oncolytic adenovirus, and the immune potentiator for the tumor treatment.

In some embodiments, the tumor includes cancers of the head, the neck, eyes, the mouth, the esophagus, the trachea, the larynx, the pharynx, the thorax, bones, lungs, the colon, the rectum, the stomach, the prostate, the bladder, the uterus, the cervix, the breast, the ovary, the testis, skins, the thyroid, blood, the lymph node, the kidney, the liver, the pancreas, the brain, or central nervous system.

In some embodiments, the tumor is the lung cancer, the liver cancer, the myeloma, the thymoma, the sarcoma, the non-Hodgkin lymphoma, the Hodgkin lymphoma, the skin cancer, the uterine cancer, the breast cancer, the pancreatic cancer, the colorectal cancer, the anal cancer, the kidney cancer, the bladder cancer, the prostate cancer, the ovarian cancer, the brain cancer, hemangioendothelioma, the head and neck cancer, the thyroid cancer, the glioma, the testicular cancer, or the gastrointestinal cancer.

In some embodiments, the tumor refers to an abnormal mass of tissue produced by uncontrolled and progressive excessive cell division, also known as a neoplasm. The tumors may be benign (non-cancerous) or malignant. Tumors cover all forms of cancer and metastasis. Tumors include all known types of tumors, including but not limited to: the breast cancer, head and neck tumors, synovial cancer, a kidney cancer, a connective tissue cancer, a melanoma, the lung cancer, a esophageal cancer, a colon cancer, a rectal cancer, the brain cancer, the liver cancer, a bone cancer, a choriocarcinoma, a gastrinoma, a pheochromocytoma, a prolactinoma, the anal cancer, a cholangiocarcinoma, the bladder cancer, a ureteral cancer, the glioma, a neuroblastoma, a meningioma, a spinal cord tumor, a osteochondroma, a chondrosarcoma, a Ewing's sarcoma, primary unknown cancer, a carcinoid, a fibrosarcoma, a Paget's disease, a cervical cancer, a gallbladder cancer, an eye cancer, a Kaposi's sarcoma, the prostate cancer, the testicular cancer, a skin squamous cell cancer, a mesothelioma, a multipoint myeloma, the ovarian cancer, a pancreatic endocrine tumor Glucagon tumor, the pancreatic cancer, a penis cancer, a pituitary cancer, a soft tissue sarcoma, a retinoblastoma, a small intestine cancer, a stomach cancer, a thymus cancer, a trophoblastic cancer, a hydatidiform mole, a endometrial cancer, a vaginal cancer, a vulva cancer, a mycosis fungoides, a insulinoma, a heart cancer, a meningeal cancer, a plurality of types of blood cancers, a peritoneal cancer, a pleural cancers, etc. Any type of tumors is covered by the present disclosure.

In some embodiments of the present disclosure, a novel mutant E1A antagonizing ubiquitination-mediated degradation (mutating lysine residues at positions 253 and 285 of the wild-type E1A to any amino acid other than lysine) is provided for the first time, which reduces degradation of the key adenovirus replication protein E1A and maintains its high-level expression, thereby promoting the oncolytic adenovirus replication and improving tumor killing effect. The mutant E1A may be applied in the preparation of the oncolytic adenovirus and the immune potentiated oncolytic adenovirus related products and has broad clinical application prospects.

The present disclosure is further elaborated below in conjunction with specific examples, which are used only for explaining the present disclosure and are not to be construed as a limitation of the present disclosure. It is understood by one of ordinary skill in the art that a wide variety of changes, modifications, substitutions, and variations can be made to these examples without departing from the principles and purposes of the present disclosure, and that the scope of the present disclosure is limited by the claims and their equivalents. The experimental methods in the following examples, for which specific conditions are not indicated, are generally performed in accordance with conventional conditions, or testing is performed in accordance with conditions recommended by the manufacturer.

### EXAMPLES

### Example 1: The Mutant E1A Antagonizing Ubiquitination-Mediated Degradation, Construction of the Oncolytic Adenovirus Using the Mutant E1A, and its Functional Validation

### 1. Experimental Methods

E1A is a key gene for the adenovirus replication, and ubiquitination-mediated degradation of E1A attenuates the replication ability of the oncolytic adenovirus and affects the oncolytic effect. There are three lysine sites on the E1A protein, namely, K208, K253, and K285. In the previous work of the present disclosure, mutant E1A with single mutant, double mutant, and triple mutant were constructed by point mutation. By co transfecting 293T cells with the E3 ubiquitin ligase TRIM35 expression plasmid and the wild-type E1A or the mutant E1A expression plasmids, an E1A ubiquitination-mediated degradation model was established. Western blot detection results indicate that mutations at K253 and K285 sites significantly inhibit ubiquitination-mediated degradation of E1A induced by E3 ubiquitin ligase TRIM35. When K253R and K285R mutations coexist, i.e., K253/285R and K208/253/285R, the degradation effect of TRIM35 on the adenovirus E1A disappears, and the K208/253/285R triple mutation does not have a better protective effect on E1A than the K253/285R double mutation, indicating that the K253/285R double mutation site is a key amino acid site for ubiquitination-mediated degradation of E1A. The specific experimental methods were as follows.
(1) On the basis of the wild-type E1A coding gene, point mutation technology was used to mutate lysine at positions 208, 253, and 285 to obtain E1A plasmids with single-site mutants, double-site mutants, and triple-site mutants.
(2) 293T cells were co-transfected with the plasmid carrying E3 ubiquitin ligase TRIM35 with V5 tag and the wild-type E1A plasmid or point mutant E1A plasmid with Flag
(3) After 24-48 hours, transfected 293T cells were collected for extracting total protein, and Western blot analysis was performed.
(4) The primary antibodies against Flag, V5, and GAPDH were incubated overnight to detect the expression of E1A and point mutant E1A.

The oncolytic adenoviruses of wild-type E1A and K253/285R mutant E1A were prepared separately. The specific preparation method was as follows.
(1) Preparation of the oncolytic adenovirus of the wild-type E1A (OAV)

The adenovirus shuttle plasmid pZD55 including wild-type adenovirus E1A and type 5 adenovirus backbone plasmid pPE3 were co-transfected into HEK293 cells to recombine the OAV.

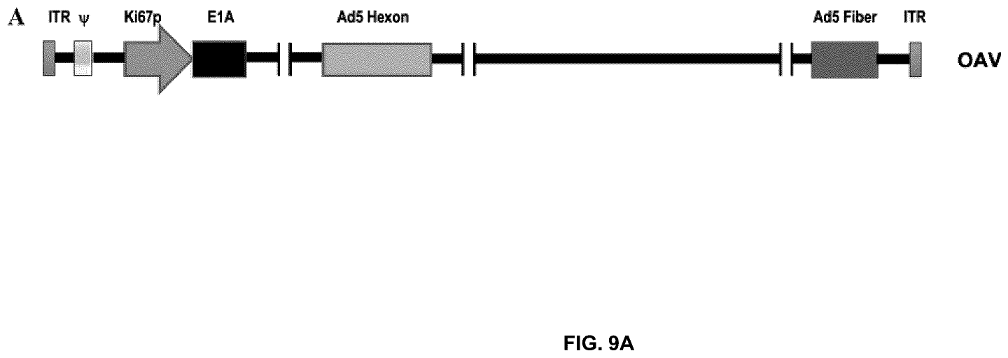

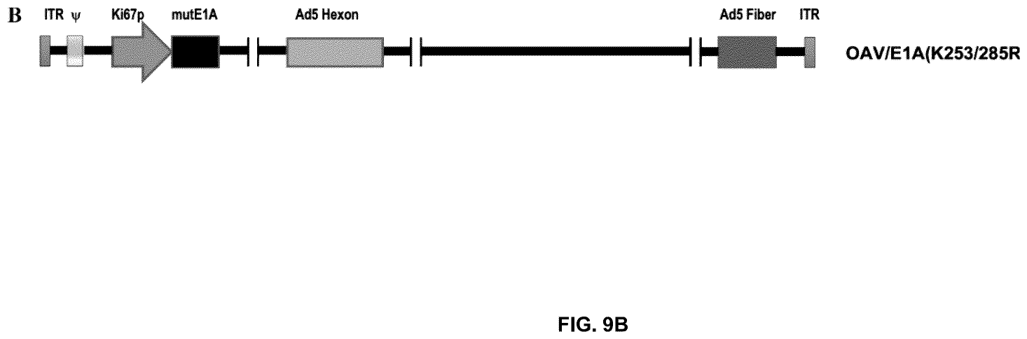

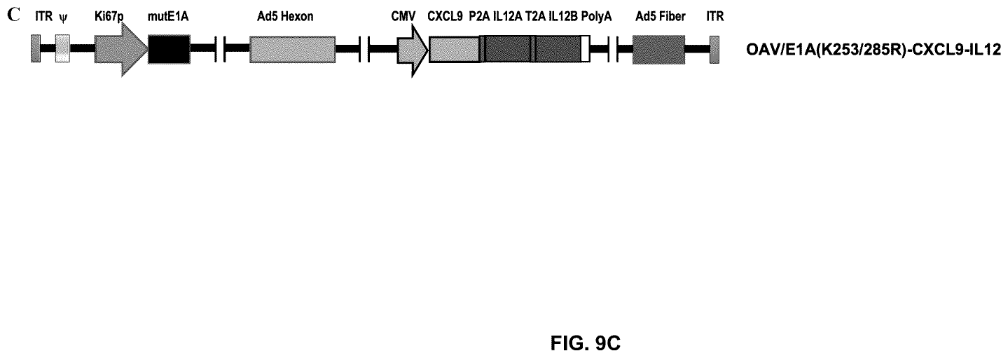

A schematic diagram of the OAV is shown in FIG. 9A. It operably includes the following elements sequentially linked: the Ki67 promoter (Ki67p), the wild-type E1A, Ad5 Hexon, and Ad5 Fiber. The amino acid sequence of the wild-type E1A is shown in SEQ ID NO:2, the nucleotide sequence encoding wild-type E1A is shown in SEQ ID NO: 1; and the nucleotide sequence of the Ki67 promoter is shown in SEQ ID NO:5.
(2) Preparation of the oncolytic adenovirus of K253/285R mutant E1A (OAV/E1A(K253/285R)).

The adenovirus shuttle plasmid pZD55 was used as a template, and the primers for site-directed mutation at the 253K and 285K sites of E1A were designed respectively, and then the plasmid pZD55-K253/285R with the 253K and 285K double-site mutation to R was obtained by site-directed mutation, and pZD55-K253/285R and the type 5 adenovirus backbone plasmid pPE3 were co-transfected into HEK293 cells to recombine the OAV/E1A(K253/285R).

A schematic diagram of the OAV/E1A(K253/285R) is shown in FIG. 9B. It operably includes the following elements sequentially linked: the Ki67 promoter (Ki67p), the mutant E1A (mutE1A), Ad5 Hexon, and Ad5 Fiber. The amino acid sequence of the mutant E1A is shown in SEQ ID NO:4, the nucleotide sequence encoding mutant E1A is shown in SEQ ID NO:3; and the nucleotide sequence of the Ki67 promoter is shown in SEQ ID NO:5.

After determining the viral titers of the OAV and the OAV/E1A(K253/285R), different tumors target cells were infected with the OAV or OAV/E1A(K253/285R) with MOI=20 to test the replication ability in different target cells. The specific experimental method was as follows.
(1) The kidney cancer 786-0 and OSRC-2 cells, the prostate cancer PC-3 and RM-1 cells, and the glioma U251 and U87 cells were collected and inoculated in six-well plates.
(2) After 24 hours, the cell culture supernatant was removed, and cells were washed twice with PBS pre-warmed to 37° C.
(3) 1 mL of virus including PBS or the OAV or the OAV/E1A(K253/285R) was added according to MOI=20 for incubating for 2 hours at 37° C.
(4) The medium including the viral fluid was removed and it was replaced with complete medium including 10% FBS, and the incubation and observation were continued for 24 hours.
(5) After 24 hours, the cell status of each group was observed under the microscope and photographed for preservation.

In the example, the tumor killing effects of the OAV and OAV/E1A(K253/285R) were detected by real-time cell analysis (RTCA). The specific experimental method was as follows.
(1) The kidney cancer 786-0 and OSRC-2 cells, the prostate cancer PC-3 cells, and the glioma U251 and U87 cells were collected and inoculated in RTCA plates at 10,000 cells per well.
(2) After 24 hours, the corresponding OAV or OAV/E1A(K253/285R) was added to the RTCA plates, and the culture was continued for observation for 2-3 days.
(3) The RTCA instrument automatically recorded the cell growth index.
(4) After 2-3 days, the RTCA was stopped and curves reflecting the cell growth index were automatically generated.

In the example, the tumor killing effects of the OAV and OAV/E1A(K253/285R) were further validated in a mouse kidney cancer subcutaneous xenograft model. The specific experimental method was as follows.

Five-week-old male Balb/c nude mice were subcutaneously inoculated with 2×10⁶/150 μL of the kidney cancer OSRC-2 cells on the right back.

The state of the mice was observed every 3 days, and their body weight was measured. One week after the tumor cells inoculation, the tumor volume was about 50-100 mm³.

Mice were randomly divided into 3 groups labeled as PBS group, OAV treatment group, and OAV/E1A(K253/285R) treatment group.

The OAV or OAV/E1A(K253/285R) viruses were diluted with PBS to 1×10⁹/150 μL and injected intratumorally at 50 μL every other day.

The state of the mice was observed every 3 days, their body weight and volume were measured.

The experiment was terminated on day 39 based on the state of the mice and the growth of the tumors, the mice were euthanized, and the tumors and major organs were taken.

Mouse tumors were weighed and fixed overnight with 4% paraformaldehyde together with the organs for immunohistochemical analysis.

### 2. Experimental Results

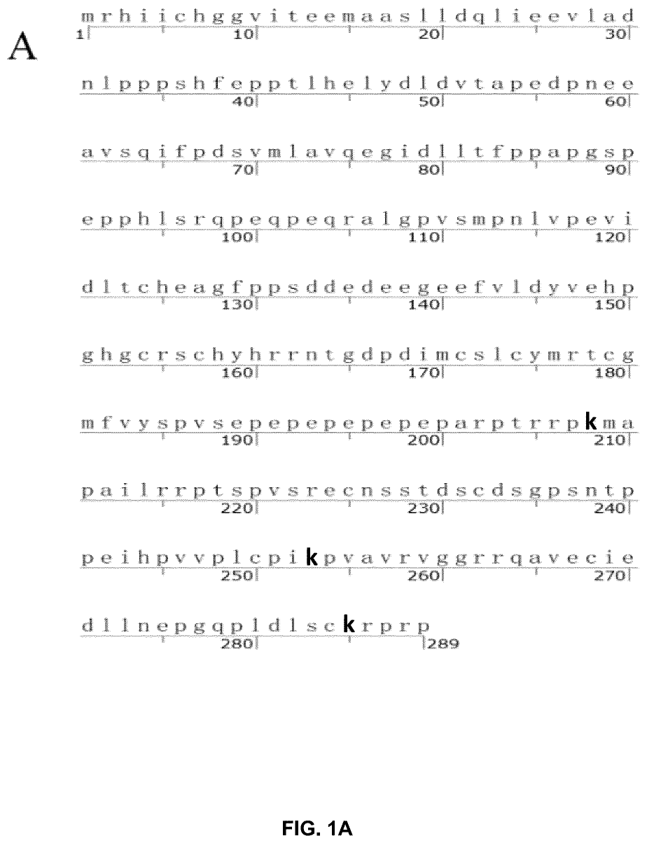

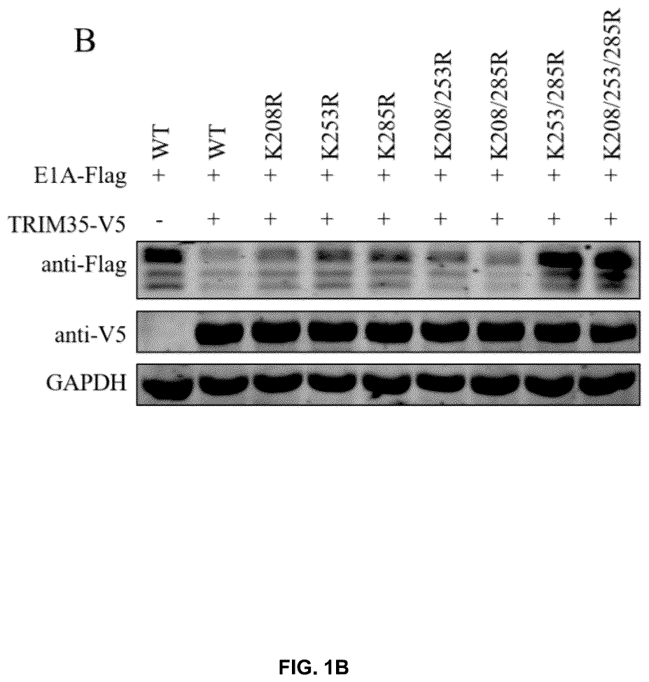

The specific mutation site of E1A and the results of Western blot analysis are shown in FIGS. 1A-1B. The bolded parts in FIG. 1A indicate mutation sites; and FIG. 1B shows an assay of E1A ubiquitination-mediated degradation (E1A point mutation antagonizing ubiquitination-mediated degradation induced by E3 ubiquitin ligase TRIM35). The amino acid mutations at positions 253 and 285 of E1A, a key gene for the adenovirus replication, reduce its ubiquitination-mediated degradation.

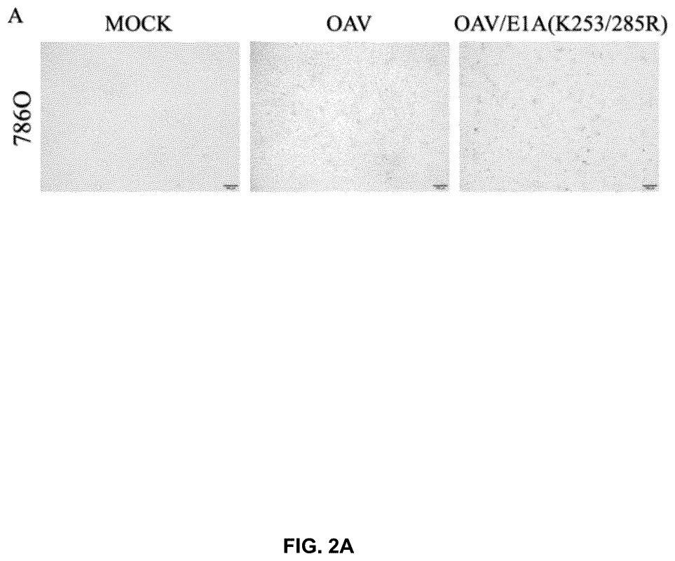

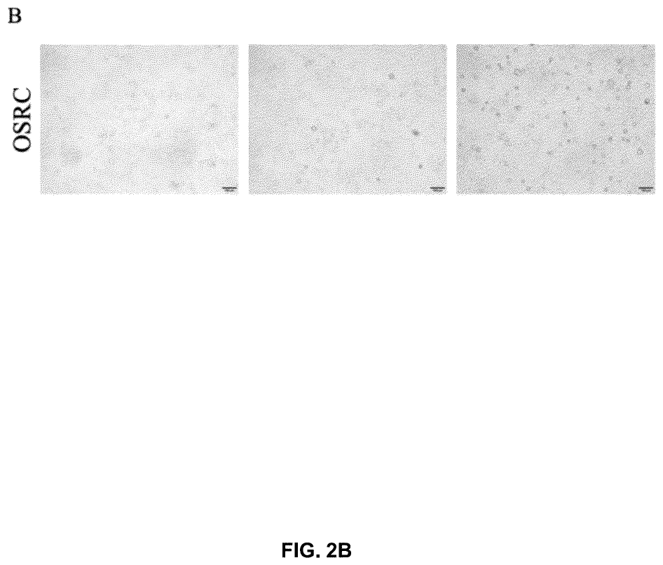

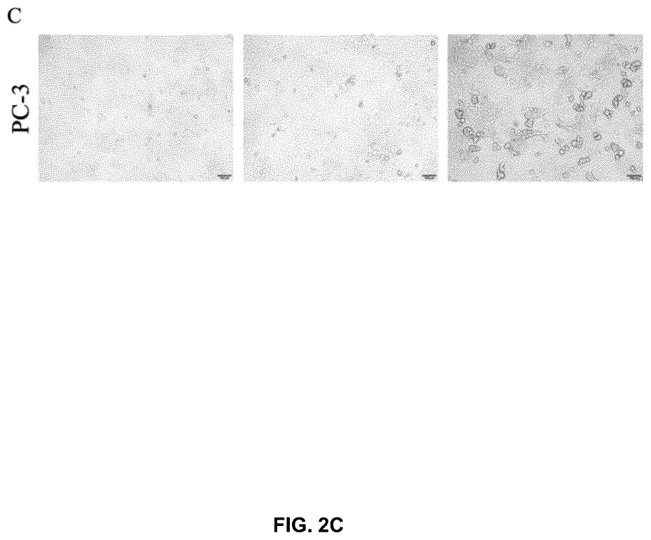

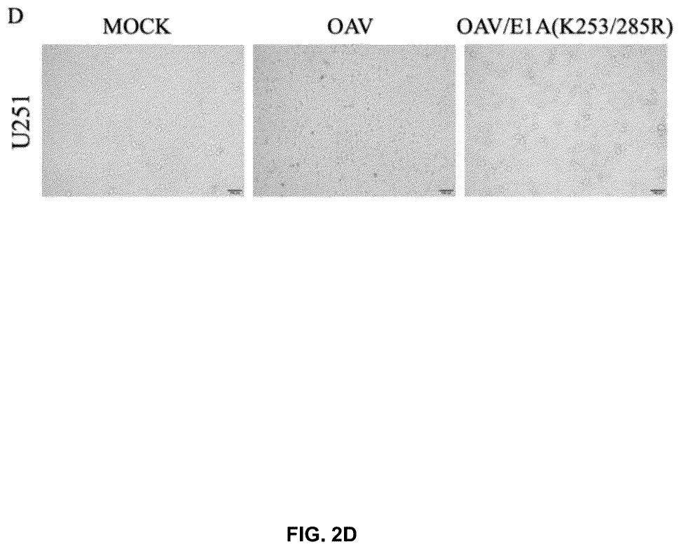

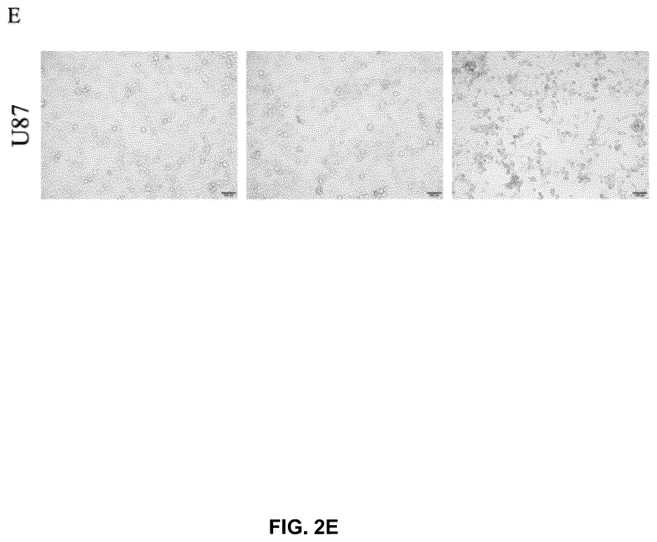

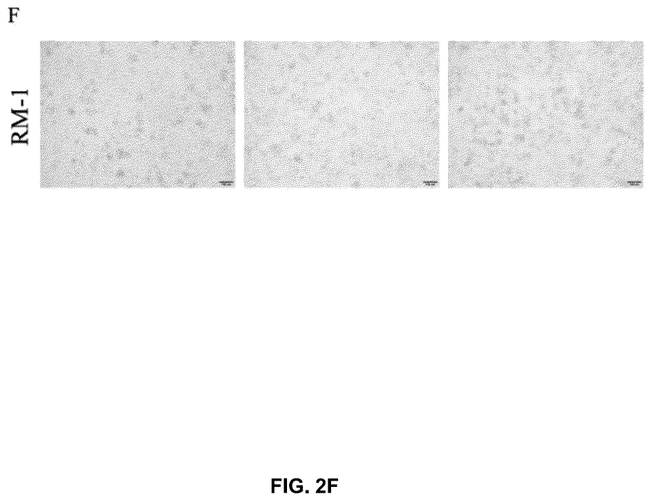

The results of cellular morphological changes and lesions after 24 h of infection with OAV and OAV/E1A(K253/285R) in different cell lines are shown in FIGS. 2A-2F. The results indicate that OAV/E1A(K253/285R) exhibits an earlier cytopathic effect and a superior oncolytic effect compared to the OAV. The result suggests that the K253/285R mutant E1A enhances replication ability of the OAV and improves its oncolytic effect.

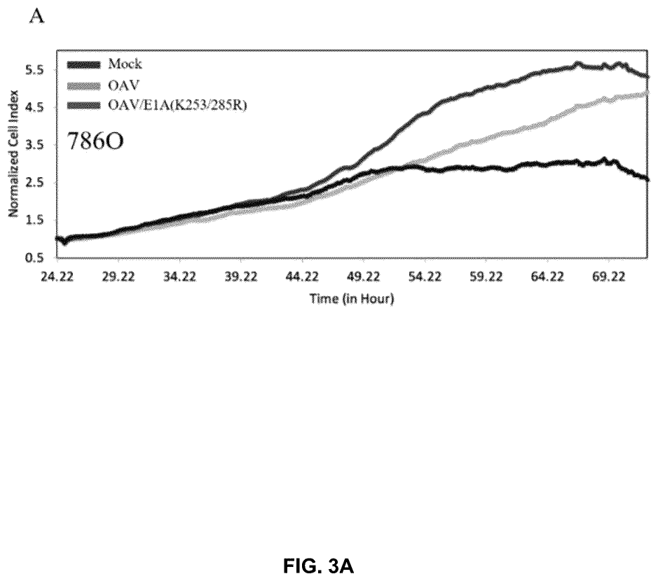

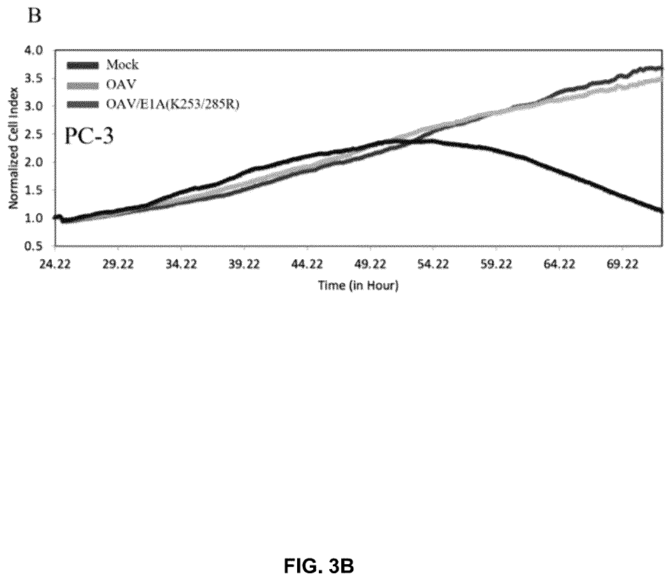

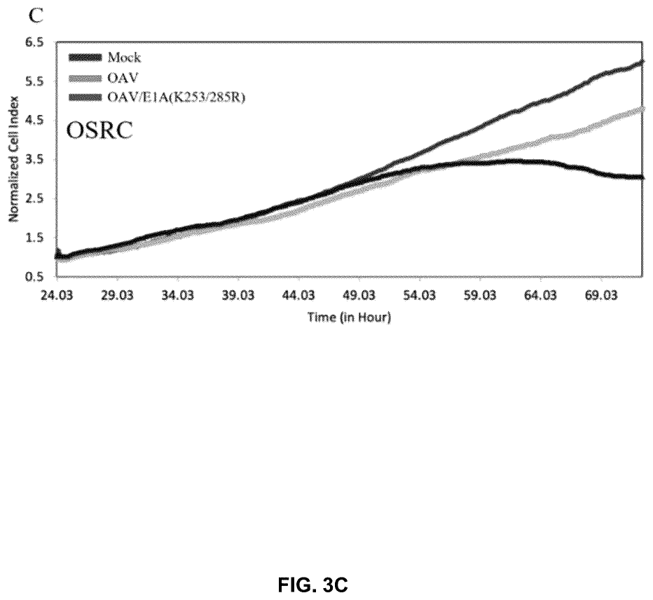

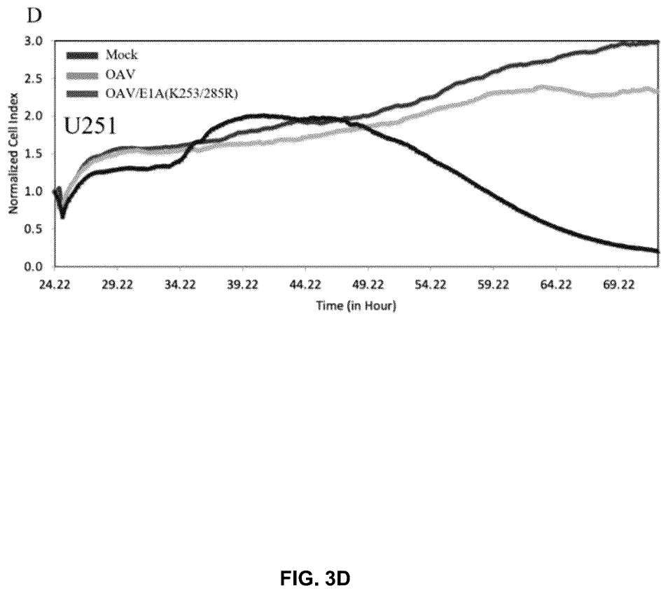

The tumor killing curves of the OAV and OAV/E1A(K253/285R) in different cell lines detected by RTCA are shown in FIGS. 3A-3D. The results indicate that in a plurality of tumor cell lines such as the kidney cancer, the prostate cancer, and the glioma, the tumor killing effect of OAV/E1A(K253/285R) is superior to that of the OAV, indicating that K253/285R mutation of E1A can enhance the replication ability of the adenovirus and enhance the tumor killing effect.

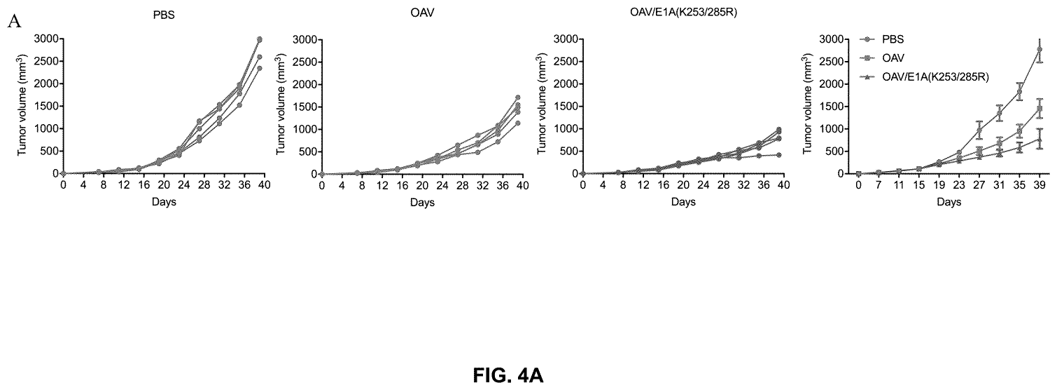

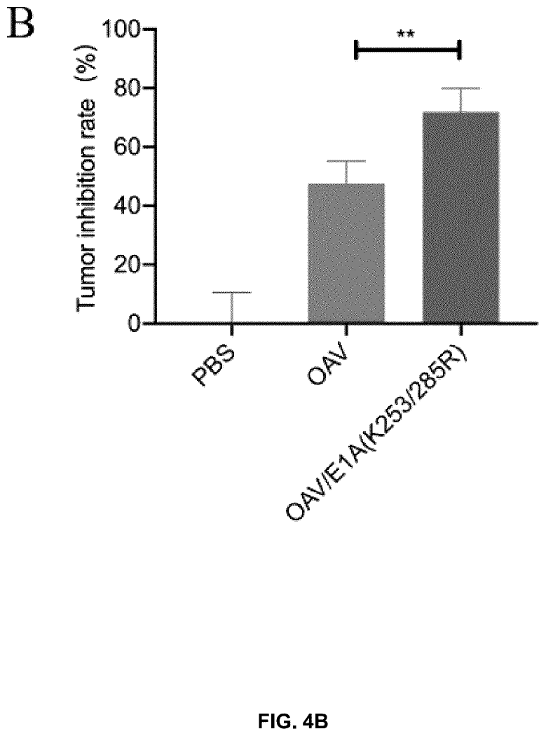

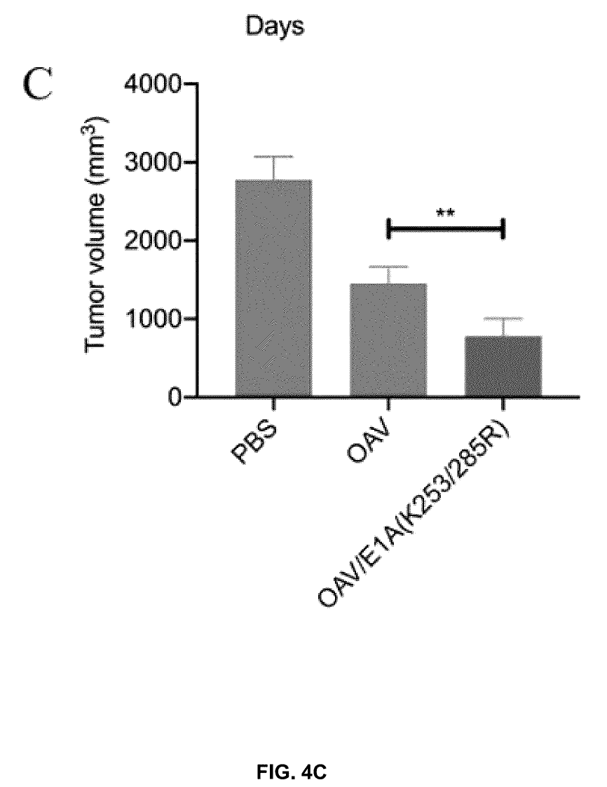

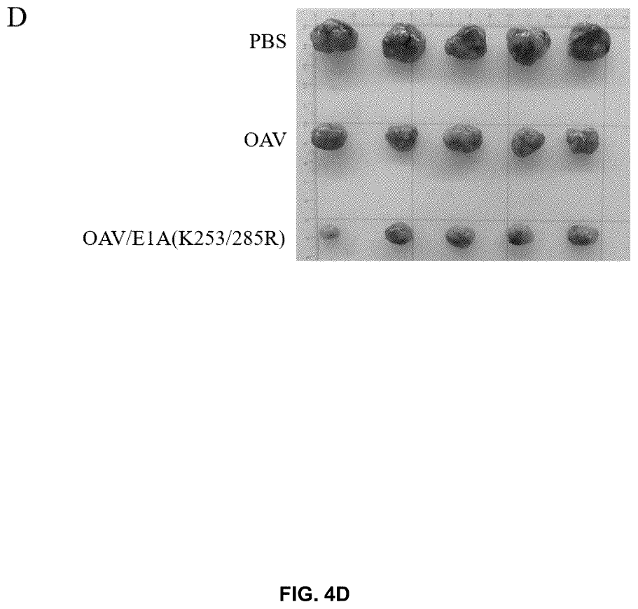

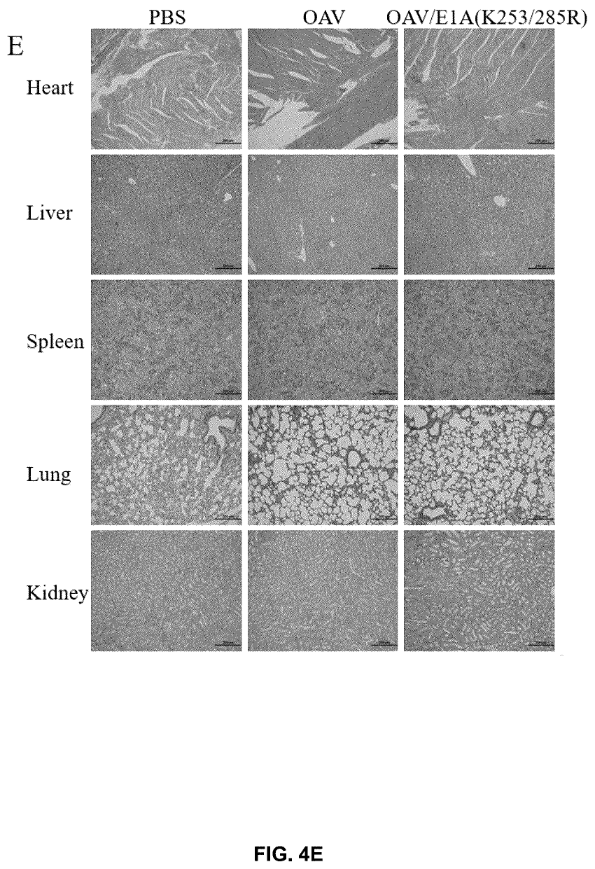

The experimental results validated in the mouse kidney cancer subcutaneous xenograft model are shown in FIGS. 4A-4E. The results indicate that the tumor inhibition effect of OAV/E1A(K253/285R) is significantly better than that of the OAV, indicating that K253/285R mutation of E1A can enhance the replication ability of the adenovirus and improve the tumor killing effect.

The above results indicate that mutations at positions 253 and 285 of the key adenovirus replication gene E1A can reduce its ubiquitination-mediated degradation, while mutations at positions 253 and 285 of the key adenovirus replication gene E1A can significantly enhance the replication ability and oncolytic effect of the oncolytic adenovirus.

### Example 2: Construction of Immune Potentiated Oncolytic Adenovirus Using the Mutant E1A and its Functional Validation

1. Experimental methods
(1) Preparation of the oncolytic adenovirus co expressing CXCL9 and IL12

The complete reading frame of CMV promoter+CXCL9-IL12+PolyA sequence with Bglll restriction sites at both ends was synthesized. The sequence was cloned into the Bglll site of the pKi67-ZD55-K253/285R vector to construct the pKi67-ZD55-K253/285R-CXCL9-IL12 plasmid. The constructed adenovirus vector pKi67-ZD55-K253/285R-CXCL9-IL12 and the type 5 adenovirus backbone plasmid pPE3 were co transfected to HEK293 cells to recombine the recombinant oncolytic adenovirus including cytokines CXCL9 and IL12 (OAV/E1A(K253/285R)-CXCL9+IL12).

The schematic diagram of the oncolytic adenovirus (OAV/E1A(K253/285R)-CXCL9+IL12) co expressing CXCL9 and IL12 is shown in FIG. 9C. It includes the following elements connected in sequence: Ki67 promoter (Ki67p), the mutant E1A (mutE1A), Ad5 Hexon, CMV promoter, CXCL9, P2A, IL-12A, T2A, IL-12B, PolvA (SV40polyA), Ad5 Fiber. The amino acid sequence of the mutant E1A is shown in SEQ ID NO: 4, and the nucleotide sequence encoding mutant E1A is shown in SEQ ID NO:3.

The nucleotide sequence of Ki67 promoter is shown in SEQ ID NO:5. The amino acid sequence of CXCL9 is shown in SEQ ID NO:7, and the nucleotide sequence encoding CXCL9 is shown in SEQ ID NO:6. The amino acid sequence of P2A is shown in SEQ ID NO: 9, and the nucleotide sequence encoding P2A is shown in SEQ ID NO:8. The nucleotide sequence of IL-12A is shown in SEQ ID NO:10. The amino acid sequence of T2A is shown in SEQ ID NO: 12, and the nucleotide sequence encoding T2A is shown in SEQ ID NO:11. The amino acid sequence of IL-12B is shown in SEQ ID NO:14, and the nucleotide sequence encoding IL-12B is shown in SEQ ID NO:13. The nucleotide sequence of SV40 polyA is shown in SEQ ID NO:15.
(2) Detection of CXCL9 and IL-12 levels in the supernatants after different target cells infected with OAV/E1A(K253/285R)-CXCL9+IL12 by ELISA
   (a) Day 1: A549, MDA-MB-231, 7860, and Huh7 cells were seeded into 10 cm dishes, 2 dishes for each type of cells, with 2×10⁶ cells per dish.
   (b) After 24 hours, the above 4 types of tumor cells were infected with OAV/E1A(K253/285R)-CXCL9+IL12 according to MOI=20, and dishes without virus were used as controls.
   (c) After continuing to culture for 24 hours, the medium in the dishes was replaced with serum-free medium. After washing the cells with PBS twice before the medium replacement, 5 mL of serum-free medium was added to each dish to continue culture.
   (d) Culture supernatants were collected after 24 hours. The supernatants were centrifuged at 12,000 rpm and stored in fractions.
   (e) The culture supernatants collected above were used in ELISA to detect the expression of CXCL9 (Biolegend, 440807) and IL12 (Beijing Yiqiao Shenzhou, KITCT011), respectively.
(3) Transwell assay for directional chemotaxis of activated T cells by supernatants from liver cancer target cells infected with OAV/E1A(K253/285R)-CXCL9+IL12
   (a) Huh7 cells were seeded into 2 dishes of 10 cm, with 2×10⁶ cells per dish.
   (b) After 24 hours, the above cells were infected with OAV/E1A(K253/285R)-CXCL9+IL12 according to MOI=20, and dishes without virus were used as controls.
   (c) After continuing to culture for 24 hours, the medium was replaced with serum-free medium. After the cells were washed twice with PBS before medium replacement, 5 mL of serum-free medium was added to each dish to continue the culture.
   (d) Culture supernatants were collected after 24 hours. The supernatants were centrifuged at 12,000 rpm and stored in fractions.
   (e) Eight 5 μm 24-well transwell chambers (with X-VIVO (500 μL) before use) were taken and placed into 24-well plates for equilibrating at 37° C. for 1-2 hours. Activated T cells were taken for counting afterward, and the upper chamber was spread with 1×106 (100 μL in X-VIVO) of activated T cells, and the lower chamber was added with 500 μL of different treatment media (DMEM 500 μL, DMEM 500 μL+CXCL9 (10 ng/ml), supernatant from Huh7 tumor cells 500 μL, and supernatant from Huh7 tumor cells infected with virus Ad5-Ki67-ZD55-mutantBC-LOXP-CXCL9+IL12 500 μL), 2 replicate wells were included per group. The number of T cells in the lower chamber was counted 3 hours later.
(4) Flow cytometry assay for promotion of proliferation of activated T cells by supernatants from liver cancer target cells infected with OAV/E1A(K253/285R)-CXCL9+IL12
   (a) Cytokine starvation of activated T cells: activated T cells were collected, after centrifuging 9×10⁶ cells, the culture medium was replaced with X-vivo medium including 10% FBS (without IL-2) for culturing for 12 hours.
   (b) Grouping treatment: cytokine starved T cells were seeded into a 24 well plate, with 3×10⁵ T cells in 500 μL of X-vivo culture medium including 10% FBS (without IL-2) per well. Grouping treatment (Blank, IL12 (20 ng/ml), Huh7-CM, Huh7-OAV-CXCL9-IL12-CM, A549-CM, A549-OAV-CXCL9-IL12-CM, where CM is cell culture medium) was performed, with 2 replicates per well, and 500 μL of supernatant from different treatment groups of tumor cells or blank culture medium was added to each well. The culture was continued for 24 hours.
   (c) Cellular EdU staining and detection: cell proliferation was detected by an EdU kit (BeyoClick™ EdU-555 Cell Proliferation Detection Reagent, BeyoClick). EdU (20 μM) was added and incubated for 4 hours, then surface-staining was performed with APC-CD3, followed by fixation and rupture of the membrane and the addition of CLICK reaction solution in the kit, and EdU was detected by flow cytometry assay.
(5) Flow cytometry assay for promotion of IFN-γ expression of T cells by supernatant from the liver cancer target cells infected with OAV/E1A(K253/285R)-CXCL9+IL12
   (a) Supernatants from tumor cells infected with OAV-CXCL9-IL12 and control supernatants were collected.
   (b) The experiment was divided into six groups: Blank, IL12 (20 ng/ml), Huh7-CM, Huh7-OAV-CXCL9-IL12-CM, A549-CM, and A549-OAV-CXCL9-IL12-CM, with three replicate wells per group, and 500 μL of tumor supernatant was added to each well of a 24-well plate, along with 5x105 activated T cells in 500 μL of X-vivo (without IL-2) medium including 10% FBS.
   (c) The culture was continued for 24 hours. Then Inomycin with a final concentration of 500 ng/ml, PMA with a final concentration of 50 ng/ml, and Brefeldin A with a final concentration of 5 ng/ml were added to the culture system, and the cells were collected after stimulation at 37° C. for 5 hours. The cells were centrifuged at 1500 rpm for 5 minutes, washed once with PBS, and incubated with the addition of APC-CD3 antibody for 30 minutes.
   (d) The cells were washed once with 1 mL of PBS and centrifuged at 1500 rpm for 5 minutes, and supernatant was discarded. 200 μL of 1×Fix fixative was added to fix cells in the dark for 40 minutes and the cells were centrifuged at 1500 rpm for 5 minutes, and supernatant was discarded. Then 500 μL of 1×Perm solution was added to the cells and the cells were centrifuged at 1500 rpm for 5 minutes, and supernatant was discarded. FITC-IFN-γ antibody was diluted with 1×Perm solution and 50 μL of antibody solution was added to each sample and incubated at 4° C. overnight.
   (e) 500 μL of 1×Perm solution was added and centrifuged at 1500 rpm for 5 minutes, the supernatant was discarded, 300 μL of PBS was added to resuspend the cells, and the cell suspension was transferred to a flow cytometry tube for analysis.
      2. Experimental results
(1) Detection of CXCL9 and IL-12 levels in supernatants after different target cells infected with OAV/E1A(K253/285R)-CXCL9+IL12 by ELISA

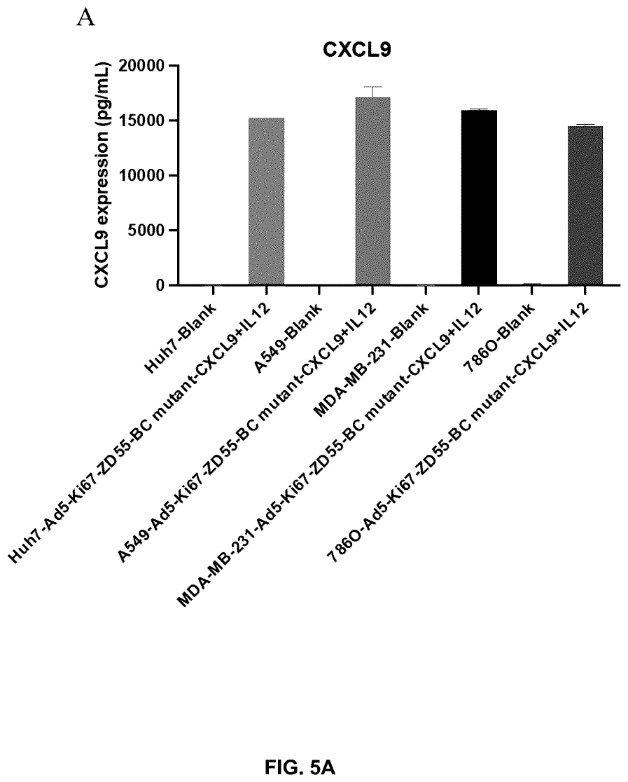

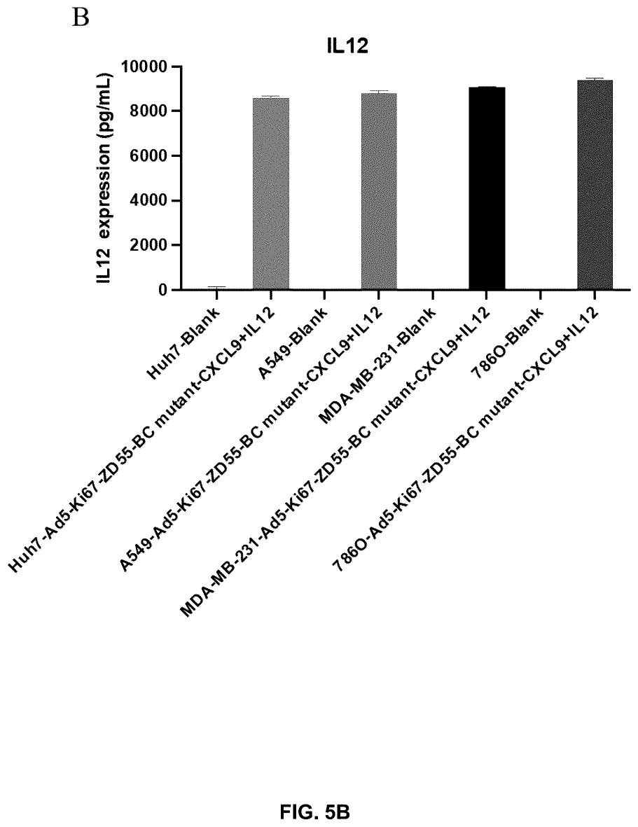

The results are shown in FIGS. 5A-5B. After the kidney cancer cells, the lung cancer cells, and the liver cancer cells infected with the immune potentiated OAV/E1A(K253/285R)-CXCL9+IL12 virus, the expression of cytokines CXCL9 and IL-12 in the supernatants was detected. The results indicate that cytokines CXCL9 and IL-12 are efficiently expressed in various tumor target cells and released into the cell culture supernatants.
(2) Transwell assay for directional chemotaxis of activated T cells by supernatants from liver cancer target cells infected with OAV/E1A(K253/285R)-CXCL9+IL12

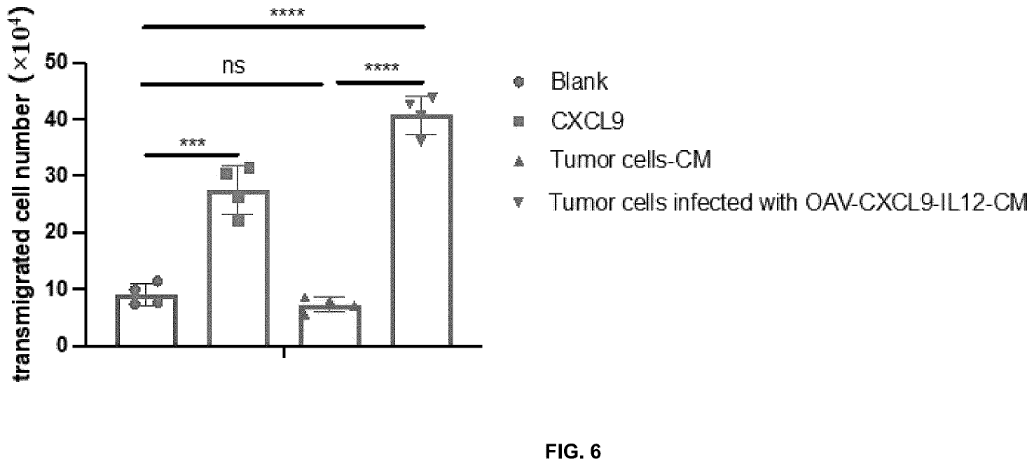

As shown in FIG. 6, after immune potentiated OAV/E1A(K253/285R)-CXCL9+IL12 virus infects liver cancer Huh-7 cells, the cell culture supernatants were collected for transwell assay. The results indicate that like the CXCL9 chemokine in the control group, the cell supernatant from the liver cancer Huh-7 cells infected with OAV/E1A(K253/285R)-CXCL9+IL12 virus can induce directional migration of activated T cells, indicating that it helps promote tumor infiltration of immune cells.

(3) Flow cytometry assay for promotion of proliferation of activated T cells by supernatants from liver cancer target cells infected with OAV/E1A(K253/285R)-CXCL9+IL12

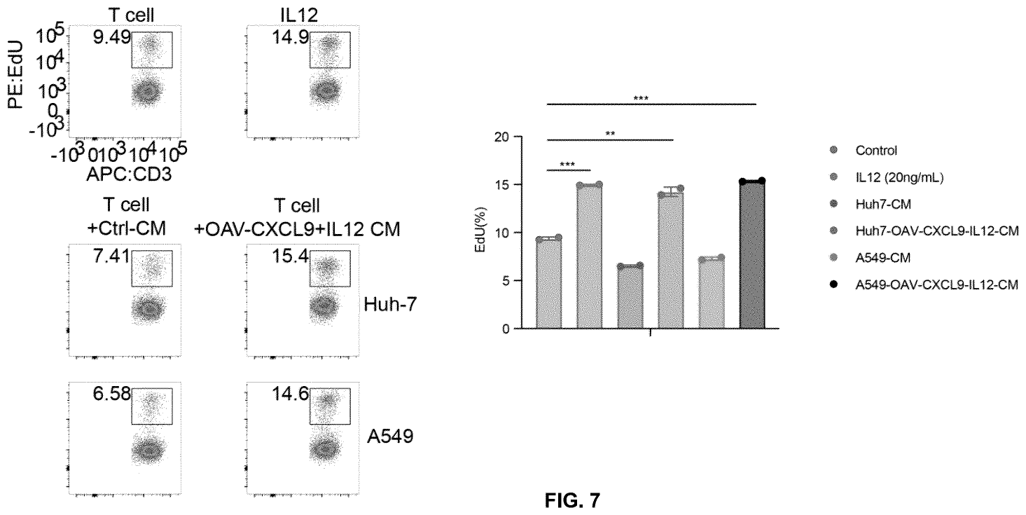

The results are shown in FIG. 7. Cell culture supernatants were collected after immune potentiated OAV/E1A(K253/285R)-CXCL9+IL12 virus infects target cells. The results indicate that cell supernatants from the target cells infected with OAV/E1A(K253/285R)-CXCL9+IL12 virus can promote the proliferation of activated T cells.
(4) Flow cytometry assay for promotion of IFN-γ expression of T cells by supernatants from liver cancer target cells infected with OAV/E1A(K253/285R)-CXCL9+IL12

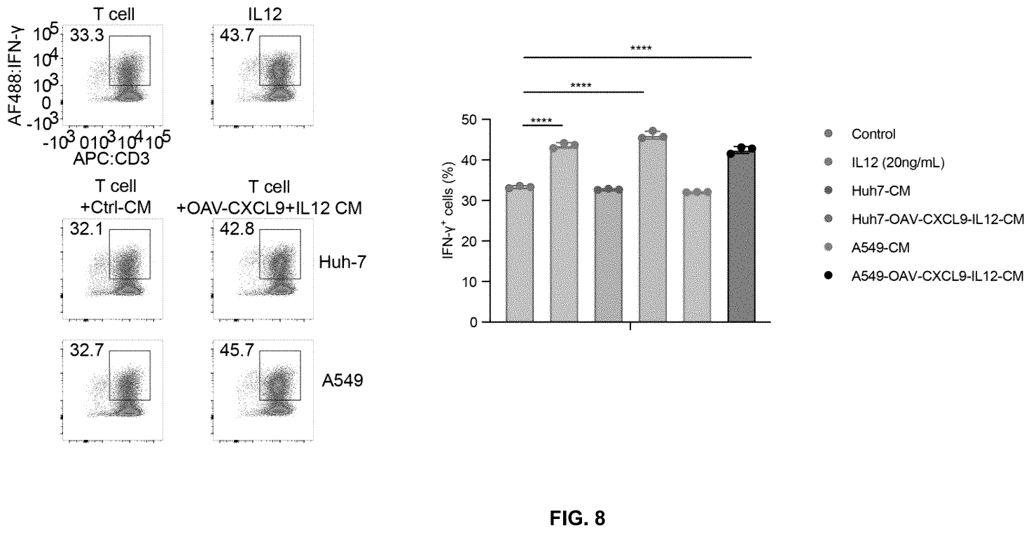

As shown in FIG. 8, after immune potentiated OAV/E1A(K253/285R)-CXCL9+IL12 virus infects target cells, the cell culture supernatants were collected. The results indicate that the cell supernatant from the target cells infected with OAV/E1A(K253/285R)-CXCL9+IL12 virus can promote the IFN-γ expression of activated T cells and enhance anti-tumor function of T cells.

The basic concepts have been described above, and it is apparent to those skilled in the art that the foregoing detailed disclosure serves only as an example and does not constitute a limitation of the present disclosure. Although not explicitly stated here, technical personnel in the field make various modifications, improvements, and revisions to the manual. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments of the present disclosure. Such as “one embodiment,” “an embodiment,” or “some embodiments” means a feature, structure, or characteristic associated with at least one embodiment of the present disclosure. Accordingly, it should be emphasized and noted that “one embodiment” or “an embodiment” referred to two or more times in the present disclosure at a plurality of locations is a term used in the present disclosure, “an embodiment” or “an alternative embodiment” in different places in the present disclosure do not necessarily refer to the same embodiment. In addition, certain features, structures, or characteristics in one or more embodiments of the present disclosure are suitably combined.

In addition, unless expressly stated in the claims, the order of the processing elements and sequences, the use of numerical letters, or the use of other names as described herein are not intended to qualify the order of the processes and methods of the present disclosure. While some embodiments of the invention that are currently considered useful are discussed in the foregoing disclosure by way of a plurality of examples, it is to be understood that such detail serves an illustrative purpose only, and that additional claims are not limited to the disclosed embodiments; rather, the claims are intended to cover all amendments and equivalent combinations that are consistent with the substance and scope of the embodiments of the present disclosure.

Similarly, it should be noted that in order to simplify the presentation of the disclosure of the present disclosure, and thereby aid in the understanding of one or more embodiments of the invention, the foregoing descriptions of embodiments of the present disclosure sometimes group a plurality of features together in a single embodiment, accompanying drawings, or a description thereof. However, the method of disclosure does not imply that the objects of the present disclosure require more features than those mentioned in the claims. Rather, claimed subject matter lies in less than all features of a single foregoing disclosed embodiment.

Numbers describing the number of components, attributes, and properties are used in some embodiments, and it is to be understood that such numbers used in the description of embodiments are modified in some examples by the modifiers “about,” “approximately,” or “substantially”. Unless otherwise noted, the terms “about,” “approximate,” or “approximately” indicate that a ±20% variation in the stated number is allowed. Correspondingly, in some embodiments, the numerical parameters used in the present disclosure and claims are approximations, which can change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present disclosure are approximations, in specific embodiments such values are set to be as precise as possible within a feasible range.

For each of the patents, patent applications, patent application disclosures, and other materials cited in the present disclosure, such as articles, books, present sheets, publications, documents, and the like, are hereby incorporated by reference in their entirety into the present disclosure. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, or use of terms in the materials appurtenant to the present disclosure and those set forth in the present disclosure, the description, definition, or use of terminology in the present disclosure shall control.

Finally, it should be understood that the embodiments described in the present disclosure are only used to illustrate the principles of the embodiments of the present disclosure. Other deformations also fall within the scope of the present disclosure. As such, alternative configurations of embodiments of the present disclosure are considered to be consistent with the teachings of the present disclosure as an example, not as a limitation. Correspondingly, the embodiments of the present disclosure are not limited to the embodiments expressly presented and described herein.

What is claimed is:

1. A mutant E1A antagonizing ubiquitination-mediated degradation, wherein the mutant E1A is obtained by mutating lysine residues at positions 253 and 285 of a wild-type E1A to any one of glycine, alanine, valine, leucine, isoleucine, proline, phenylalanine, tyrosine, tryptophan, serine, threonine, cysteine, methionine, asparagine, glutamine, aspartic acid, glutamic acid, arginine, or histidine.

2. An oncolytic adenovirus vector comprising the mutant E1A of claim 1, wherein a backbone sequence of the oncolytic adenovirus vector is an adenovirus genome.

3. The oncolytic adenovirus vector of claim 2, wherein a serotype of an adenovirus is Ad5, Ad3, Ad9, Ad11, Ad12, Ad34, or Ad37.

4. A potentiated oncolytic adenovirus vector comprising the mutant E1A of claim 1, wherein the potentiated oncolytic adenovirus vector further comprises:
a sequence of at least one of an immunomodulatory factor, an immune checkpoint inhibitor, an immunomodulatory molecule, an antigenic molecule, an enzyme, or an small interfering RNA (siRNA).

5. The potentiated oncolytic adenovirus vector of claim 4, wherein the immunomodulatory factor is selected from a group consisting of an interleukin (IL), an interferon (INF), a tumor necrosis factor, a colony stimulating factor, and a chemokine.

6. The potentiated oncolytic adenovirus vector of claim 5, wherein the interleukin is selected from at least one of a group consisting of IL-1, IL-18, IL-33, IL-36, IL-37, IL-38, IL-2, IL-4, IL-13, IL-15, IL-21, IL-6, IL-11, IL-27, IL-31, OSM, LIF, CNTF, CT-1, CLCF1, IL-12, IL-23, IL-27, IL-35, IL-10, IL-19, IL-20, IL-22, IL-24, IL-26, IL-28, IL-29, IL-17, and IL-25.

7. The potentiated oncolytic adenovirus vector of claim 5, wherein the interferon is selected from at least one of a group consisting of IFN-α, IFN-β, and IFN-γ.

8. The potentiated oncolytic adenovirus vector of claim 5, wherein the chemokine is selected from at least one of a group consisting of CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8 (IL-8), CXCL9, CXCL10, CXCL11, CXCL12, CXCL13, CXCL14, CXCL15, CXCL16, CXCL17, XCL1, XCL2, and CX3CL1.

9. The potentiated oncolytic adenovirus vector of claim 4, wherein the potentiated oncolytic adenovirus vector comprises a sequence of the mutant E1A, a CXCL9 sequence, and an IL-12 sequence.

10. The potentiated oncolytic adenovirus vector of claim 9, wherein a serotype of an adenovirus is Ad5, Ad3, Ad9, Ad11, Ad12, Ad34, or Ad37.

11. An oncolytic adenovirus, wherein the oncolytic adenovirus is obtained by packaging the oncolytic adenovirus vector of claim 2.

12. A potentiated oncolytic adenovirus, wherein the potentiated oncolytic adenovirus is obtained by packaging the potentiated oncolytic adenovirus vector of claim 4.

13. A pharmaceutical composition, comprising a therapeutically effective amount of the oncolytic adenovirus of claim 11.

14. A pharmaceutical composition, comprising a therapeutically effective amount of the potentiated oncolytic adenovirus of claim 12.

15. A biological agent, comprising a therapeutically effective amount of the oncolytic adenovirus of claim 11 and a pharmaceutically acceptable carrier or excipient.

16. A biological agent, comprising a therapeutically effective amount of the potentiated oncolytic adenovirus of claim 12 and a pharmaceutically acceptable carrier or excipient.

17. A method for inhibiting tumor progression or reducing tumor volume in a subject, or treating a tumor in the subject, comprising:
administering to the subject with a therapeutically effective amount of the oncolytic adenovirus of claim 11, or a therapeutically effective amount of a pharmaceutical composition comprising the oncolytic adenovirus, or a therapeutically effective amount of a biological agent comprising the oncolytic adenovirus and a pharmaceutically acceptable carrier or excipient.

18. The method of claim 17, wherein the tumor is a lung cancer, a liver cancer, a myeloma, a thymoma, a sarcoma, a non-Hodgkin lymphoma, a Hodgkin lymphoma, a skin cancer, a uterine cancer, a breast cancer, a pancreatic cancer, a colorectal cancer, an anal cancer, a kidney cancer, a bladder cancer, a prostate cancer, an ovarian cancer, a brain cancer, hemangioendothelioma, a head and neck cancer, a thyroid cancer, a glioma, a testicular cancer, or a gastrointestinal cancer.

19. A method for inhibiting tumor progression or reducing tumor volume in a subject, or treating a tumor in the subject, comprising:
administering to the subject with a therapeutically effective amount of the potentiated oncolytic adenovirus of claim 12, a therapeutically effective amount of a pharmaceutical composition comprising the potentiated oncolytic adenovirus, or a therapeutically effective amount of a biological agent comprising the potentiated oncolytic adenovirus and a pharmaceutically acceptable carrier or excipient.

20. The method of claim 19, wherein the tumor is a lung cancer, a liver cancer, a myeloma, a thymoma, a sarcoma, a non-Hodgkin lymphoma, a Hodgkin lymphoma, a skin cancer, a uterine cancer, a breast cancer, a pancreatic cancer, a colorectal cancer, an anal cancer, a kidney cancer, a bladder cancer, a prostate cancer, an ovarian cancer, a brain cancer, hemangioendothelioma, a head and neck cancer, a thyroid cancer, a glioma, a testicular cancer, or a gastrointestinal cancer.
